(19) United States
(12) Patent Application Publication
(10) Pub. No.: US 2004/0029167 A1
(43) Pub. Date: Feb. 12, 2004

(54) Inducible COMT_II promoter, chimeric gene containing same and plants transformed therewith

(76) Inventors: Bernard Fritig, Souffelweyersheim (FR); Valerie Toquin, Morlaix (FR); Pierrette Geoffroy, Stresbourg (FR); Michel Legrand, Pfettisheim (FR); Serge Kauffmann, Strasbourg (FR)

Correspondence Address: BAKER & BOTTS, 30 ROCKEFELLER PLAZA, NEW YORK, NY, 10112

(21) Appl. No.: 10/633,840

(22) Filed: Aug. 4, 2003

Related U.S. Application Data
(62) Division of application No. 09/937,204, filed on Dec. 13, 2001.
Application No. 09/937,204 is a 371 of international application No. PCT/FR00/00714, filed on Mar. 22, 2000.

(30) Foreign Application Priority Data
Mar. 22, 1999 (FR) 99/03700
Jun. 11, 1999 (FR) 99/07646

(51) Int. Cl. C12Q 1/68; C07H 21/04; C12N 9/22; A01H 1/00; C12N 15/82; C12N 5/04

(52) U.S. Cl. 435/6; 435/69.1; 435/199; 435/320.1; 435/419; 800/279; 536/23.2

(57) ABSTRACT

The invention concerns a novel regulating COMTII promoter sequence inducible in response to a mechanical or chemical injury, or in response to aggression by a pathogenic agent, in particular bacterial, fungal or viral, or by an insect or a threadworm. The invention also concerns a chimera gene (or expression cassette) comprising the inventive regulating promoter sequence controlling the expression of a heterologous coding sequence and a host organism comprising said chimera gene, transformed plants containing it and the seeds of said transformed plants.

### CROSS REFERENCE TO RELATED APPLICATIONS

This application is a divisional of pending U.S. patent application Ser. No. 09/937,204, filed on Sep. 21, 2001, which is a U.S. national stage filing of International Patent Application Ser. No. PCT/FR00/00714, filed Mar. 22, 2000 and published in English on Sep. 28, 2000 as WO00/56897, which claims priority to French Patent Appl. Ser. No. 99/03,700, filed Mar. 22, 1999 and French Patent Appl. Ser. No. 99/07,646, filed Jun. 11, 1999, the contents of which are incorporated herein in their entireties.

The present invention relates to a novel regulatory promoter sequence which is inducible in response to a mechanical or chemical injury or in response to an attack by a pathogenic agent, in particular a bacterial, fungal or viral agent, or by an insect or a nematode.

The present invention also relates to a chimeric gene (or expression cassette) comprising the regulatory promoter sequence according to the invention which controls the expression of a heterologous coding sequence, heterologous meaning herein a coding sequence different from the native coding sequence.

The present invention also relates to a host organism comprising said chimeric gene, the transformed plants comprising it and the seeds (grains) of said transformed plants.

It is known from the state of the art that certain genes, which are silent in the absence of an attack are activated only by an attack which can be mechanical as well as chemical and/or in response to an attack from a pathogenic agent, an insect or a nematode. Such genes and their activation factors are in particular described in U.S. Pat. No. 5,670,349.

These various methods of defense are generally linked to regulation of the expression of certain genes involved in the defense mechanisms of plants by induction of their regulatory promoter sequences. Several inducible regulatory sequences are known, such as the promoters of phenylalanine ammonia lyase (PAL), of HMG-CoA reductase (HMG), of chitinases, of glucanases, of proteinase inhibitors (PIs), of PR1 family genes, of nopaline synthase (NOS) or of the vspB gene, all these promoters being recalled with the references of the corresponding publications via table 3 of U.S. Pat. No. 5,670,349. The HMG2 promoter described in this same U.S. Pat. No. 5,670,349 is also known, as is the apple beta-galactosidase (ABG1) promoter or the apple aminocyclopropane carboxylate synthase (ACC synthase) promoter described in patent application WO 98/45445.

The present invention relates to a novel nucleic acid fragment, in particular an isolated fragment, comprising an inducible plant promoter (or regulatory promoter sequence), said inducible promoter consisting of the promoter of a plant class II O-methyltransferase (hereinafter COMT II) gene.

Plant class II O-methyltransferase genes, including the class II caffeic acid O-methyltransferase gene are silent (inactive) in the absence of any attack since unattacked plants do not express it, or at least express it at levels which are undetectable with the usual analytical methods. Thus, the COMT II message is undetectable using the Northern blot technique in various tissues of a nontreated healthy plant, such as for example tobacco (Pellegrini et al., 1993). This COMT II and its promoter are activated (or induced) by injuries, viral infections, attacks from UV rays or chemical attacks by various products such as benzothiazole (BTH), methyl jasmonate or elicitors of plant origin, such as pectin.

Advantageously, the isolated nucleic acid fragment according to the invention consists of a plant COMT II promoter.

According to the invention, the term “plant COMT II” is intended to mean any plant OMT which is not expressed in nontreated healthy plants but which is expressed subsequent to a mechanical or chemical attack or an attack by a pathogen, an insect or a nematode.

According to the invention, the expression “plant of origin of the COMT II” is intended to mean any differentiated multicellular organism capable of photosynthesis, whether it is monocotyledonous or dicotyledonous, such as for example rice, wheat, barley, sunflower, maize, tobacco, rapeseed, soybean or Arabidopsis thaliana.

According to a particular embodiment of the invention, the COMT II is a dicotyledonous plant COMT, preferably a tobacco COMT.

According to the invention, the term “promoter” is intended to mean the noncoding region of a gene involved in the attachment of RNA polymerase and other factors which are responsible for initiating and modulating transcription, leading to the production of an RNA transcript. It is more particularly any sequence 5′ of the translation initiation site, or start codon (ATG), of the coding sequence of the COMT II, which allows the transcription and the expression of said coding sequence.

The promoter according to the invention advantageously comprises a sequence of more than 600 nucleotides upstream of the COMT II ATG, preferably of more than 700, of more than 800 or even of more than 900 nucleotides upstream of the ATG, more preferentially of more than 1000 nucleotides upstream of the ATG, even more preferentially of more than 1200 nucleotides upstream of the ATG. The promoters comprising more than 1500 nucleotides upstream of the COMT II ATG also form part of the present invention.

The promoter comprises a transcription initiation site. The transcription initiation site is generally located less than 100 nucleotides upstream of the ATG, advantageously approximately 90 nucleotides upstream.

Advantageously, the 3′ end of the COMT II promoter according to the invention is located between the transcription initiation site and the ATG. Preferably, the 3′ end of the COMT II promoter is located between 10 and 50 nucleotides downstream of the transcription initiation site, more preferentially between 20 and 40 nucleotides downstream, even more preferentially between 20 and 30 nucleotides downstream.

The COMT II promoter according to the invention also comprises at least one TATA box and at least one CAT box. The TATA box is generally located less than 50 nucleotides upstream of the transcription initiation site, approximately 40 nucleotides upstream. The CAT box is generally located less than 100 nucleotides upstream of the transcription initiation site, preferably approximately 100 nucleotides and/or 80 nucleotides upstream. Advantageously, the promoter comprises two CAT boxes.

The promoter according to the invention also comprises regulatory elements involved in the expression of phenylpropanoid metabolism genes and genes associated with defense, in particular at least one A box and/or at least one L box and/or at least one inverted L box and/or at least one P box and/or at least one inverted W box. The A box comprises the following sequence CCGTCC. It is generally located less than 410 nucleotides from the transcription initiation site. The L box comprises the following sequence CTTCAACAACCAACC. It is generally located less than 180 nucleotides from the transcription initiation site. The first inverted L box comprises the following sequence GTTAGGTGAAG. It is generally located less than 1000 nucleotides upstream of the transcription initiation site. Advantageously, the promoter according to the invention comprises two inverted L boxes, one approximately 970 nucleotides upstream of the transcription initiation site and the other 440 nucleotides upstream. The second inverted L box comprises the following sequence TGTTAGGTGTGTGTTT. The P box comprises the following sequence CACACCAACTCCCA. It is generally located less than 750 nucleotides upstream of the transcription initiation site. The inverted W box comprises the following sequence GGTCAA. It is generally located less than 1200 nucleotides upstream of the transcription initiation site. Advantageously, the promoter according to the invention comprises two inverted W boxes, one approximately 1110 nucleotides upstream and the other approximately 210 nucleotides upstream.

The promoter according to the invention also comprises at least one E box and/or at least one G box and/or at least one GT box. The E box comprises the following sequence TTCCATCAAG. It is generally located less than 110 nucleotides upstream of the transcription initiation site. The G box comprises the following sequence CCACGT. It is generally located less than 600 nucleotides upstream of the transcription initiation site. The GT box comprises the following sequence GGTTAA. It is generally located less than 450 nucleotides upstream of the transcription initiation site. Advantageously, the promoter according to the invention comprises two GT boxes, one approximately 400 nucleotides upstream and the other approximately 280 nucleotides upstream.

According to an advantageous embodiment of the invention, the promoter according to the invention consists of the tobacco COMT II promoter defined by the nucleotide sequence upstream of the ATG, represented by sequence identifier 1 (SEQ ID NO 1). Preferably, the tobacco COMT II promoter comprises the sequence between nucleotides 557 and 1795 of SEQ ID NO 1, the sequences capable of hybridizing selectively to said sequences and the sequences homologous thereto. Preferentially, the tobacco COMT II promoter comprises the sequence between nucleotides 557 and 889 of SEQ ID NO 1, the sequences capable of hybridizing selectively to said sequences and the sequences homologous thereto. The inducible tobacco COMT II promoter is activated by injuries, viral infections, attacks from UV rays or chemical attacks by various products such as benzothiazole (BTH), methyl jasmonate or elicitors of plant origin, such as pectin. The functional study of the promoter has made it possible to identify the regions involved in the induction of the promoter under various conditions. The fragment comprising the sequence between nucleotides 698 and 1365 is necessary and sufficient to confer sensitivity and induction by injury, by TMV, megaspermine, pectin and chitin. Preferentially, the tobacco COMT II promoter therefore comprises the sequence between nucleotides 698 and 1365 or SEQ ID NO 1, the sequences capable of hybridizing selectively to said sequences and the sequences homologous thereto. This fragment may be combined with another promoter in order to induce the expression of a gene by injury, by TMV, megaspermine, pectin or chitin. This fragment may thus be combined with the minimum CaMV 35S RNA promoter. A subject of the present invention is therefore also a chimeric promoter comprising the sequence between nucleotides 698 and 1365 of SEQ ID NO 1, the sequences capable of hybridizing selectively to said sequences and the sequences homologous thereto. The analysis of the response of the promoter with respect to methyl jasmonate and UV rays indicates that the fragment comprising the sequence between nucleotides 815 and 1795 of SEQ ID NO 1 is functional. In an advantageous embodiment, the tobacco COMT II promoter therefore comprises the sequence between nucleotides 815 and 1795 of SEQ ID NO 1, the sequences capable of hybridizing selectively to said sequences and the sequences homologous thereto.

According to the invention, the expression “sequence capable of hybridizing selectively” is intended to mean the sequences which hybridize with the above sequences at a level which is significantly higher than the background noise. The background noise may be linked to the hybridization of other DNA sequences which are present, in particular of other cDNAs present in a cDNA library. The level of the signal generated by the interaction between the sequence capable of hybridizing selectively and the sequences defined by the sequence IDs above according to the invention is generally 10 times, preferably 100 times, more intense than that of the interaction of the other DNA sequences generating the background noise. The level of interaction can be measured, for example, by labeling the probe with radioactive elements such as ³²P. Selective hybridization is generally obtained by using very severe medium conditions (for example 0.03 M NaCL and 0.03 M sodium citrate at approximately 50° C.-60° C.). The hybridization can, of course, be carried out according to the usual methods of the state of the art (in particular Sambrook et al., 1989, Molecular Cloning: A Laboratory Manual).

According to the invention, the term “homologous sequence” is intended to mean any sequence which comprises more than 70% homology, preferentially more than 80% homology, even more preferentially more than 90% homology, and which conserves the functional elements of the COMT II which confer upon it its inducible promoter properties. The methods for measuring and identifying homologies between nucleic acid sequences are well known to those skilled in the art. Use may be made, for example, of the PILEUP or BLAST programs (in particular Altschul et al., 1993, J. Mol. Evol. 36: 290-300; Altschul et al., 1990, J. Mol. Biol. 215: 403-10).

The isolation, cloning and characterization of the COMT II promoters from the COMT II genes are carried out according to the usual experimental methods of those skilled in the art for isolating, cloning and characterizing a promoter, which are thoroughly described in the literature.

The isolation and cloning of a COMT II gene are carried out by analyzing a genomic library prepared from the DNA of the plant of interest. The genomic DNA is cleaved with one or more suitable restriction enzymes and introduced into a suitable vector so as to constitute, using methods known to those skilled in the art, a library containing all of the genomic DNA of the plant (Ausubel et al., 1998; Sambrook et al., 1989).

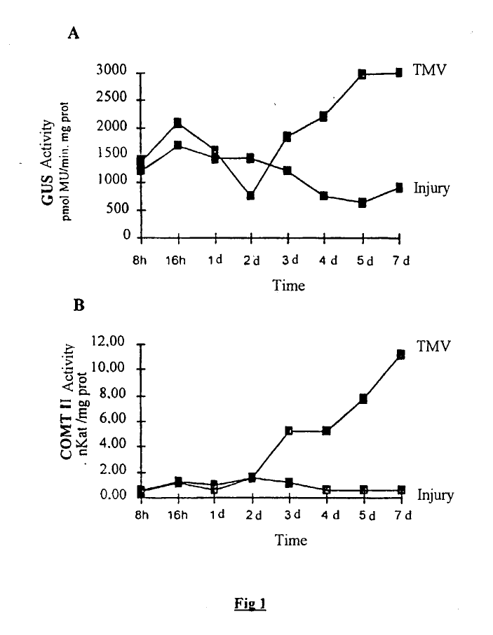

The clone(s) containing a COMT II gene is (are) isolated using a nucleotide probe. The sequence of the probe is either deduced from the protein sequence if the enzyme has been purified (by monitoring its activity, for example) or prepared from a cDNA clone derived from a library. This cDNA library is prepared from MRNA extracted from tissues treated so as to induce the expression of the COMT II gene (by injury, infection or chemical treatment as described in the examples or FIGS. 1-5). The cDNA library is then screened with antibodies directed against a COMT II protein (from tobacco for example) or with a nucleotide probe deduced from the COMT II protein of the plant under consideration or deduced from the sequences conserved in plant COMTs. The cDNA thus isolated is characterized by its nucleotide sequence or by the enzymatic activity of the recombinant protein obtained after expression of the cDNA in a prokaryotic or eukaryotic organism.

The noncoding sequences of the cDNA (3′ and/or 5′) are used to select, by PCR under very selective conditions, the genomic clone(s) containing the COMT II gene expressed during the treatment used to construct the cDNA library. The promoter sequences are then be isolated by PCR or any other suitable method well known to those skilled in the art.

Based on the information contained in the present patent application for the tobacco COMT II promoter, those skilled in the art will be able to identify other COMT II promoters from other plant species once the COMT II gene has been identified and cloned according to the usual methods, in particular those described above.

The present invention also relates to a chimeric gene (or expression cassette) which is functional in plant cells and the plants comprising, in the direction of transcription, a regulatory sequence in 5′, a coding sequence and a regulatory sequence in 3′, the regulatory sequence in 5′ comprising a COMT II promoter according to the invention defined previously.

According to the invention, the term “plant cell” is intended to mean any cell which is derived from a plant and which can constitute undifferentiated tissues such as calluses or differentiated tissues such as embryos, parts of plants, plants or seeds.

According to the invention, the term “plant” is intended to mean any differentiated multicellular organism capable of photosynthesis, in particular monocotyledons or dicotyledons, more particularly crop plants which may or may not be intended for animal or human food, such as maize, wheat, rapeseed, soybean, rice, sugar cane, beetroot, tobacco, cotton etc.

As a regulatory sequence in 5′, use may be made of the COMT II promoter according to the invention alone or combined with at least part of a promoter of a gene which is naturally expressed in plants, in particular a promoter which is expressed especially in the leaves of plants, such as for example “constitutive” promoters of bacterial, viral or plant origin, or alternatively “light-dependent” promoters, such as that of a plant ribulose-biscarboxylase/oxygenase (RuBisCO) small subunit gene, or any suitable known promoter which may be used. Among the promoters of plant origin, mention will be made of the histone promoters as described in application EP 0 507 698 or the rice actin promoter (U.S. Pat. No. 5,641,876). Among the promoters of a plant virus gene, mention will be made of that of the cauliflower mosaic virus (CAMV 19S or 35S) or the circovirus promoter (AU 689 311).

The COMT II promoter according to the invention may also be used in combination with at least part of a promoter specific for particular regions or tissues of plants, and more particularly grain-specific promoters ([22] Datla, R et al., Biotechnology Ann, Rev. (1997) 3, 269-296), in particular the napin promoter (EP 255 378), the phaseolin promoter, the glutenin promoter, the helianthinin promoter (WO 92/17580), the albumin promoter (WO 98/45460) or the oelosin promoter (WO 98/45461).

According to the invention, use may also be made, in combination with the COMT II promoter according to the invention, of other regulatory sequences, which are located between the promoter and the coding sequence, such as transcription activators (enhancers), for instance the translation activator of the tobacco mosaic virus (TMV) described in application WO 87/07644 or of the tobacco etch virus (TEV) described by Carrington & Freed, for example.

As a regulatory terminator or polyadenylation sequence, use may be made of any corresponding sequence of bacterial origin, such as for example the nos terminator of Agrobacterium tumefaciens, or of plant origin, such as for example. a histone terminator as described in application EP 0 633 317.

The coding sequence of the chimeric gene according to the invention comprises a coding sequence for a reporter gene, such as the GUS coding sequence, or a coding sequence for a protein of interest. With respect to the method of induction of the promoter according to the invention, injury, viral infection or response to elicitors, the protein of interest is advantageously a protein which confers on the plants properties of resistance to diseases or to insects.

Among the proteins or peptides of interest which confer novel properties of resistance to diseases, mention will be made in particular of chitinases, glucanases, oxalate oxydase, all these proteins and their coding sequences being widely described in the literature, or antibacterial and/or antifungal peptides, in particular cysteine-rich peptides of less than 100 amino acids, such as plant thionins or defensins, and more particularly lytic peptides of all origins comprising one or more disulfide bridges between the cysteines and regions comprising basic amino acids, in particular the following lytic peptides: androctonin (WO 97/30082 and PCT/FR98/01814, filed on Aug. 18, 1998) or drosomycin (PCT/FR98/01462, filed on Jul. 8, 1998).

According to a preferential embodiment of the invention, the protein or peptide of interest is chosen from fungal elicitor peptides, in particular elicitins (Kamoun et al., 1993; Panabières et al., 1995). Preferentially, the fungal elicitor peptide is megaspermine. Megaspermine and its coding sequence are represented by sequence identifier no. 13 (SEQ ID 13). More preferentially, the chimeric gene according to the invention comprising, in the direction of transcription, a regulatory sequence in 5′ comprising a COMT II promoter and a coding sequence for megaspermine comprises the DNA sequence represented by sequence identifier no. 14 (SEQ ID 14).

Among the proteins of interest which confer novel properties of resistance to insects, mention will be made more particularly of the Bt proteins which are widely described in the literature and well known to those skilled in the art. Mention will also be made of proteins extracted from bacteria such as Photorabdus (WO 97/17432 & WO 98/08932).

The present invention also relates to a chimeric gene (or expression cassette) which is functional in plant cells and the plants comprising, in the direction of transcription, a regulatory sequence in 5′, a coding sequence for an elicitor and a regulatory sequence in 3′, the regulatory sequence in 5′ comprising an inducible promoter.

Preferentially, the elicitor is an elicitin, more preferentially megaspermine as defined above.

The inducible promoter is advantageously chosen from the promoters of phenylalanine ammonia lyase (PAL), of HMG-CoA reductase (HMG), of chitinases, of glucanases, of proteinase inhibitors (PIs), of PR1 family genes, of nopaline synthase (nos) or of the vspB gene (U.S. Pat. No. 5,670,349, table 3), the HMG2 promoter (U.S. Pat. No. 5,670,349), the apple beta-galactosidase (ABG1) promoter or the apple aminocyclopropane carboxylate synthase (ACC synthase) promoter (WO 98/45445).

The present invention also relates to a cloning and/or expression vector for transforming plant cells or plants containing at least one chimeric gene as defined above. This vector comprises, besides the chimeric gene above, at least one origin of replication. This vector may consist of a plasmid, a cosmid, a bacteriophage or a virus, transformed by introducing the chimeric gene according to the invention. Such transformation vectors are well known to those skilled in the art and widely described in the literature. For transforming plant cells or plants, it will in particular be a virus which can be used for transforming developed plants and which also contains its own replication and expression elements. Preferentially, the vector for transforming plant cells or plants according to the invention is a plasmid.

For transforming plant cells or plants, the chimeric gene according to the invention may be used in combination with a selection marker gene, either in the same vector, the two genes being combined in a convergent, divergent or co-linear manner, or in two vectors used simultaneously for transforming the host organism. Such marker genes and their use for transforming plants are well known to those skilled in the art and widely described in the literature.

Among the genes encoding selection markers, mention may be made of the genes for resistance to antibiotics, the genes for tolerance to herbicides (bialaphos, glyphosate or isoxazoles), genes encoding easily identifiable reporter enzymes, such as the GUS enzyme, and genes encoding pigments or enzymes which regulate the production of pigments in the transformed cells. Such selection marker genes are in particular described in patent applications EP 242 236, EP 242 246, GB 2 197 653, WO 91/02071, WO 95/06128, WO 96/38567 or WO 97/04103.

A subject of the invention is also a method for transforming plant cells by integrating into the genome of said plant cells at least one chimeric gene as defined above, it being possible to obtain this transformation by any suitable known means, thoroughly described in the specialized literature and in particular the references cited in the present application, more particularly with the vector according to the invention.

A series of methods consists in bombarding cells, protoplasts or tissues with particles to which the DNA sequences are attached. Another series of methods consists in using, as a means of transfer into the plant, a chimeric gene inserted into an Agrobacterium tumefaciens Ti plasmid or an Agrobacterium rhizogenes Ri plasmid. Other methods may be used, such as microinjection or electroporation, or alternatively direct precipitation with PEG. Those skilled in the art will choose the suitable method as a function of the nature of the host organism, in particular of the plant cell or of the plant.

A subject of the present invention is also the plant cells or plants which are transformed and contain a chimeric gene defined above.

The subject of the present invention is also the plants containing transformed cells, in particular the plants regenerated from the transformed cells. The regeneration is obtained using any suitable method, which depends on the nature of the species, as described, for example, in the references above. For the methods for transforming plant cells and for regenerating plants, mention will be made in particular of the following patents and patent applications: U.S. Pat. Nos. 4,459,355, 4,536,475, 5,464,763, 5,177,010, 5,187,073, EP 267,159, EP 604 662, EP 672 752, U.S. Pat. Nos. 4,945,050, 5,036,006, 5,100,792, 5,371,014, 5,478,744, 5,179,022, 5,565,346, 5,484,956, 5,508,468, 5,538,877, 5,554,798, 5,489,520, 5,510,318, 5,204,253, 5,405,765, EP 442 174, EP 486 233, EP 486 234, EP 539 563, EP 674 725, WO 91/02071 and WO 95/06128.

The present invention also relates to the transformed plants derived from culturing and/or crossing the regenerated plants above, and also the grains of transformed plants.

The transformed plants which may be obtained according to the invention can be of the monocotyledon type, such as, for example, cereals, sugar cane, rice and maize, or the dicotyledon type, such as, for example, tobacco, soybean, rapeseed, cotton, sunflower, beetroot, clover, etc.

The transformed plants according to the invention may contain other genes of interest, which confer on the plants novel agronomic properties. Among the genes which confer novel agronomic properties on the transformed plants, mention may be made of the genes which confer tolerance to certain herbicides, those which confer tolerance to certain insects and those which confer tolerance to certain diseases. Such genes are in particular described in patent applications WO 91/02071 and WO 95/06128. Mention may also be made of the genes which modify the composition of the modified plants, in particular the content and quality of certain essential fatty acids (EP 666 918) or the content and quality of the proteins, in particular in the leaves and/or the grains of said plants. Mention will be made in particular of the genes encoding proteins enriched in sulfur-containing amino acids ([1]; WO 98/20133; WO 97/41239; WO 95/31554; WO 94/20828; WO 92/14822). The function of these proteins enriched in sulfur-containing amino acids will also be to trap and store excess cysteine and/or methionine, making it possible to avoid the possible problems of toxicity linked to an overproduction of these sulfur-containing amino acids, by trapping them. Mention may also be made of genes encoding peptides rich in sulfur-containing amino acids, and more particularly in cysteines, said peptides also having antibacterial and/or antifungal activity. Mention will be made more particularly of plant defensins as well as lytic peptides of any origin, and more particularly the following lytic peptides: androctonin (WO 97/30082 and PCT/FR98/01814, filed on Aug. 18, 1998) or drosomycin (PCT/FR98/01462, filed on Jul. 8, 1998).

These other genes of interest may be combined with the chimeric gene according to the invention either by conventionally crossing two plants, each containing one of the genes (the first containing the chimeric gene according to the invention and the second containing the gene encoding the protein of interest) or by transforming plant cells of a plant containing the gene encoding the protein of interest with the chimeric gene according to the invention.

The examples hereinafter make it possible to illustrate the invention without, however, seeking to limit the scope thereof.

All the methods or operations described below in these examples are given by way of examples and correspond to a choice made from the various methods available for achieving the same result. This choice has no bearing on the quality of the result and, consequently, any suitable method may be used by those skilled in the art to achieve the same result. Most of the methods for engineering DNA fragments are described in “Current Protocols in Molecular Biology” volumes 1 and 2, Ausubel F. M. et al or in Sambrook et al 1989.

Description of FIG. 1: Kinetics of activities GUS (1A) corresponding to the construct COMT II promoter (−1215 to +24)/GUS and COMT II (1B) during viral infection (TMV) or during injury.

### EXAMPLE 1

### Isolation of the Class II COMT Gene

The screening of a tobacco genomic library made it possible to isolate 6 different clones containing class II COMT (COMT II) genes. The latter were first characterized by their restriction profiles, which revealed a certain heterogeneity among the various clones.

The COMT IIs form a multigenic family composed of six to seven genes, just one of which is transcribed in defense reactions since a single cDNA type was characterized in a library produced from leaves inoculated with the tobacco mosaic virus (TMV) (Pellegrini et al. 1993). In order to identify the clone(s) containing the gene expressed during the defense reactions, PCR reactions were carried out using primers derived from the 3′ noncoding region of the cDNA. Under highly selective conditions, a single clone is then amplified. The amplification products were sequenced. The sequences obtained exhibited total homology with those of the 3′ noncoding regions of the cDNA.

### EXAMPLE 2

### Analysis of the Sequences of the Class II COMT Gene Promoter

The genomic clone selected was subcloned into a bacterial vector (puc 18) and represents a 14 kb insert, 9 kb of which are located upstream of the ATG of the COMT II gene.

The transcription initiation site was determined using the primer extension technique and it is located 90 nucleotides from the transcription initiation site.

The promoter was sequenced over a length of 1771 kilobases. Nonspecific elements common to eukaryotic genes and involved in the initiation of transcription, such as the TATA box and the CAT box, were found in the COMT II promoter (SEQ ID NO 1). Regulation sites were demonstrated by comparing the promoter sequences of the COMT II gene with those of genes involved in defense mechanisms. The COMT II promoter contains elements specific for the phenylpropanoid metabolism genes involved in the response to stress, such as the three boxes P, A, and L (initially identified in the parsley PAL gene) (SEQ ID NO 1). These three boxes are involved in the response to elicitors and the P and L boxes are also involved in the response to UV rays. The E box initially identified in the parsley CCoAOMT gene and extremely conserved in the phenylpropanoid metalolism genes also plays a role in the response to elicitors.

The COMT II promoter also has elements which play an important role in the induction of PR genes by elicitors, such as the W box (SEQ ID NO 1).

General regulatory elements are found in the COMT II promoter, such as the G and GT boxes and the activator element of the SV40 simiens virus (SEQ ID NO 1). The G box is an element present in a large variety of plant promoters. The G box, associated with cis specific elements, is involved in the regulation of many genes which respond to various physiological and environmental signals. The promoter region, which is responsible for the regulation of genes by methyl jasmonate, consists of a G box associated with sequences rich in C nucleotides. A similar organization is found in the promoter of genes specifically induced during injury. The L boxes, present in the COMT II promoter, may be involved in this type of interaction since they are units rich in C nucleotides.

The GT boxes, represented several times in the promoters, appear to play a role in modulating the expression of certain plant genes, either as an activator or as a repressor.

### EXAMPLE 3

### Functional Analysis of the Promoter Regions of the COMT II Gene

The functional analysis of the COMT II promoter was carried out by stable expression trans-genesis. The transgene was obtained by transcriptional fusion between the promoter and a reporter gene, the GUS (β-glucuronidase) gene. Four constructs corresponding to various deletions of the promoter were produced in order to specify the nature of the important promoter sequences responsible for the regulation of the gene. These constructs correspond to the promoter sequences from −1215 to +24 base pairs (bp), from −420 to +24 bp, from −313 to +24 bp and from −121 to +24 bp (with respect to the +1 transcription site), 557 to 1795, 1352 to 1795, 1459 to 1795 and 1651 to 1795, respectively, on SEQ ID NO 1, introduced into the pBi101 (Clontech) vector, upstream of the GUS gene.

The various constructs were introduced, via Agrobacterium tumefaciens, into the genome of the plants. A population of about ten transformed tobacco plants was regenerated for each construct. The level of the expression of the transgene was determined by assaying the enzymatic activity and with histochemical tests. In parallel, the expression of the COMT II genes was analyzed by measuring the activity of the corresponding enzymes.

Results:

The GUS activity was assayed in these plants under various conditions for inducing defense reactions, with a fungal elicitor injected into the leaves (megaspermine) or after exposure to UV rays. The results obtained are given in tables 1 and 2 below. The GUS activity is expressed in pmol MU/min. mg. For table 1, the control (C) consists of the infiltration of water into the leaves of the transformed plants. Control plants transformed with an empty vector had an activity of approximately 10-30 pmol/min.mg. For table 2, the control (C) corresponds to the basal GUS activity in the nontreated plants.

TABLE 1
Expression of the GUS activity
corresponding to the various COMT II/GUS constructs:
induction with megaspermine

GUS Activity

| COMT II/GUS Construct | C | Megaspermine |
|---|---|---|
| COMT II −1215 to +24 | 150 | 1,150 |
| COMT II −420 to +24 | 2 | 6 |
| COMT II −313 to +24 | 0.4 | 0.8 |
| COMT II −121 to +24 | 0.2 | 0.8 |

TABLE 2
Expression of the GUS activity
corresponding to various COMT II/GUS constructs:
induction with UV rays

GUS Activity

| COMT II/GUS Construct | C | UV |
|---|---|---|
| COMT II −1215 to +24 | 90 | 900 |
| COMT II −420 to +24 | 10 | 12 |
| COMT II −313 to +24 | 10 | 11 |
| COMT II −121 to +24 | 11 | 10 |

These results show that the size of the promoter must be greater than 600 bp, in this case 1239 bp, in order to allow the induction and strong expression of the GUS gene. The deletions of the promoters corresponding to the constructs (−420 to +24), from (−313 to +24) and from (−121 to +24) cause total loss of expression of the GUS gene under all the conditions assayed. The activity of the GUS gene under the control of the 1239 bp promoter is 1000 times greater than that observed for the other constructs.

### Activation of the COMT II Promoter by Injury and Methyl Jasmonate and by Elicitors of Various Origin and Nature

The transgenic plants containing the construct COMT II (−1215 to +24)/GUS were treated with various chemical products, regulators of defense reactions, with methyl-2,6-dichloroisonicotinic (INA) and salicylic acid (SA), with fungal elicitors such as glucans or fragments of chitin and an elicitor of plant origin, such as pectin. The GUS activity was measured in the leaves 16 h after infiltration of these compounds and the results are given in table 3 below. The GUS activity is expressed in pmol MU/min.mg. The control (C) corresponds to the basal GUS activity in the nontreated transformed plants.

TABLE 3
Induction of the GUS activity by
elicitors

| | GUS Activity | Induction % |
|---|---|---|
| C | 600 | 100 |
| H20 | 1,200 | 200 |
| SA (1 mM) | 1,200 | 200 |
| INA (1 mM) | 1,700 | 200 |
| Chitins (100 μg/ml) | 1,200 | 200 |
| Glucans (200 μg/ml) | 1,400 | 200 |
| Pectins (200 μg/ml) | 3,700 | 600 |

The greatest increase in the GUS activity (about 3-fold) is obtained in the plants infiltrated with pectin fragments, compared to the control.

The induction of the 1239 bp promoter was studied during injury or after treatment with methyl jasmonate (molecule which plays a role in defense response signaling during injury) or with benzothiadiazole (BTH) (chemical SAR-inducer). The GUS activity is measured 16 h after treatment. The results are given in table 4 below.

TABLE 4
Induction of the GUS activity by
various compounds and stress

| | Induction of the GUS activity % |
|---|---|
| C | 100 |
| BTH | 250 |
| Injury | 600 |
| Methyl jasmonate | 1,450 |
| UV | 1,000 |

The control (C) corresponds to the GUS activity of the nontreated plants. The promoter is activated by the various treatments.

The induction factors range from 2.5 (BTH) to 14.5 (methyl jasmonate).

### Activation of the COMT II Promoter During Viral Infection

In tobacco, TMV inoculation requires the production of microinjuries in the leaves which allow entry of the virus and its multiplication. The GUS activity and the COMT II activity were measured in the injured leaves and in the leaves inoculated with the virus. The results (FIG. 1) show that the GUS gene under the control of the COMT II promoter has induction kinetics which are identical to those of the endogenous COMT II gene, monitored by measuring the catalytic activity of the corresponding proteins. The COMT II promoter is induced early by the injury and exhibits maximum activity at 16 h. The same activity peak is observed at 16 h during the viral infection and is due to the injury to the leaves caused by the inoculation of the virus. In the inoculated leaves, the GUS activity is greatly stimulated from the 3rd day and increases up to the 7th day. The local and systemic induction of the promoter during the viral infection was measured 3 and 7 days after the TMV inoculation. The GUS activities expressed in pmol MU/min.mg are given in table 5 and represent a mean of the values obtained in 9 transformants. The control C corresponds to the GUS activity of the nontreated plants.

TABLE 5
Local and systemic (SAR) induction
of the GUS activity

| | GUS Activity |
|---|---|
| C | 700 |
| Leaves inoculated after 3 days | 3,800 |
| Leaves inoculated after 7 days | 7,100 |
| SAR leaves after 7 days | 2,700 |

These results show an 1100% induction of the promoter at 7 days in the inoculated leaves. The GUS activity measured at 7 days in the SAR leaves is weaker but very significant. However, in the noninoculated leaves, the induction factors calculated from the GUS activities are greater than those obtained from the COMT II activities (table 6). This is due, firstly, to the fact that the GUS activity assay is more sensitive than that for COMT II and, secondly, to the fact that the GUS protein is extremely stable and that the GUS activity measured corresponds to an accumulation of this protein after treatment. The comparison of the GUS and COMT II activities is given in table 6 below. The noninoculated leaves in which systemic acquired resistance develops are termed “SAR leaves”.

TABLE 6
Induction factors for the GUS and
COMT II activities 3 and 7 days after inoculation with
TMV, in the infected leaves and the SAR leaves

| | Induction of GUS | | Induction of COMT II | |
|---|---|---|---|---|
| | activities | | activities | |
| | Inoculated | | Inoculated | |
| | leaves | SAR leaves | leaves | SAR leaves |
| 3 days | 5.8 | | 5.8 | |
| 7 days | 11 | 3.9 | 15 | 1.8 |

### Histochemical Analysis of the GUS Activity During Viral Infection and After Injury

A histochemical analysis of the GUS activity in the leaves inoculated with TMV, 7 days after virosis, shows that the expression of the GUS gene is located in the cells surrounding the site of infection. Transverse sections of the leaves at the level of the lesions were prepared in order to determine the cell types involved, and show that the induction of the GUS gene is not tissue specific but involves all the cells around the lesions.

The histochemical analysis of the expression of the GUS gene in injured leaves, 2 days after treatment, shows induction of the GUS gene in the uninjured tissues surrounding the sites of injury by pricking or using forceps. These results imply that a signal is emitted from the injured tissues to the intact tissues, inducing systemic expression of the GUS gene. The methyl jasmonate synthesized in the injured tissues may induce, from a distance, the GUS gene, since exogenous application of methyl jasmonate induces GUS activity. The histochemical test carried out on leaves derived from 35S/GUS transgenic plants is the positive control for the experiment. Transverse sections of injured leaves also show that all cell types are induced by the injury.

### Activity of Promoter Fragments Which are Variable in Size and Between Bases −1215 and −406

List of the constructs containing the GUS reporter gene under the control of the promoter sequences between −1215 and −406 (with respect to the +1 transcription initiation site):

Control Constructs: GUS reporter gene lacking promoter GUS reporter gene under the control of the minimum CaMV 35S RNA promoter (p min).

COMT II/GUS Constructs Used:

COMT II −956 to +24

COMT II −937 to +24

COMT II −882 to +24

COMT II −746 to +24

COMT II −676 to +24

COMT II −560 to +24

COMT II −435 to +24

COMT II −1073 to −406+minimum CaMV 35S RNA promoter (p min).

These constructs correspond, respectively, to fragments 815 to 1795, 834 to 1795, 889 to 1795, 1025 to 1795, 1095 to 1795, 1211 to 1795, 1336 to 1795 and 698 to 1365 of SEQ ID NO 1.

The functional analysis of these various constructs was carried out on transgenic tobacco plants in stable expression.

The studies were first carried out on a population of approximately 20 vitroplants which had allowed the selection of 3 to 7 lines per construct, which have analyzed at the age of 20 to 45 days after transfer under glass.

TABLE 7
Induction by injury, methyl jasmonate, UV rays and TMV of the GUS
activity in tobacco vitroplants transformed with the various
COMT II/GUS constructs.

| | Number of plants responding to the | | | | |
|---|---|---|---|---|---|
| | signal/total number of plants analyzed | | | | |
| | Control | | | | |
| COMT | kept | | Methyl | | |
| II/GUS constructs | alive | Injury | jasmonate | UV | TMV |
| COMT II −1215 to + | 4/15 | 3/7 | 9/15 | 10/15 | 7/15 |
| 24 | | | | | |
| COMT II −956 to + | 4/20 | 6/15 | 9/20 | 15/20 | 7/20 |
| 24 | | | | | |
| COMT II −937 to + | 0/20 | 5/10 | 0/20 | 0/20 | 8/20 |
| 24 | | | | | |
| COMT II −882 to + | 0/20 | 0/17 | 0/20 | 0/20 | 0/20 |
| 24 | | | | | |
| COMT II −746 to + | 0/20 | 0/4 | 0/20 | 0/20 | 0/20 |
| 24 | | | | | |
| COMT II −676 to + | 0/20 | 0/13 | 0/20 | 0/20 | 0/20 |
| 24 | | | | | |
| COMT II −560 to + | 0/20 | 0/8 | 0/20 | 0/20 | 0/20 |
| 24 | | | | | |
| COMT II −435 to + | 0/20 | 0/5 | 0/20 | 0/20 | 0/20 |
| 24 | | | | | |
| COMT II −1073 to − | 1/20 | 9/27 | 0/20 | 0/20 | 10/20 |
| 406 | | | | | |
| pSI 101 | 0/20 | 0/5 | 0/20 | 0/20 | 0/20 |
| (without promoter) | | | | | |
| p min/GTJS | 0/20 | 0/11 | 0/20 | 0/20 | 0/20 |

In order to test the response to injury, vitroplant leaves (aged from 4 to 6 weeks after planting out) were injured using forceps and the GUS histochemical activity was revealed 16 hours after injury.

In order to test the response to infection, vitroplant leaves were inoculated with a solution of TMV and the GUS activity was revealed 7 days after inoculation.

The treatments with UV rays and methyl jasmonate, and also the noninduced control, were carried out on vitroplant leaves kept alive in water for 16 hours.

TABLE 8
Induction of the GUS activity by
fragments of pectin or chitin or by megaspermine, in
tobacco plants cultivated under glass and transformed
with the various COMT II/GUS constructs

| | Number of plants responding to the | | |
|---|---|---|---|
| | signal/total number of plants analyzed | | |
| COMT II/GUS constructs | Pectins | Chitins | Megaspermine |
| COMT II −1215 to +24 | 5/6 | 4/6 | 4/6 |
| COMT II −956 to +24 | 4/5 | 4/5 | 4/5 |
| COMT II −937 to +24 | 1/3 | 1/3 | 2/3 |
| COMT II −882 to +24 | 0/5 | 0/5 | 0/5 |
| COMT II −746 to +24 | 0/5 | 0/5 | 0/5 |
| COMT II −676 to +24 | 0/5 | 0/5 | 0/5 |
| COMT II −560 to +24 | 0/5 | 0/5 | 0/5 |
| COMT II −435 to +24 | 0/5 | 0/5 | 0/5 |
| COMT II −1073 to −406 | 5/5 | 4/5 | 5/5 |
| pBI | 0/5 | 0/5 | 0/5 |
| (without promoter) | | | |
| p min/GUS | 0/6 | 0/6 | 0/6 |

The deletions corresponding to the constructs (−882 to +24), (−746 to +24), (−676 to +24), (−560 to +24) and (−435 to +24) cause complete loss of the inducibility of the COMT II promoter for all of the stresses studied in tables 7 and 8. This indicates that the sequences required for the activity of the promoter are located between bases −1215 and −882 with respect to the transcription initiation site.

In the cases of the induction of the promoter by injury, TMV and megaspermine, and also by fragments of pectin and of chitin, only the promoter fragments (−956 to +24), (−937 to +24) and (−1073 to −406) allow the expression of the GUS activity. In addition, the fragment (−1073 to −406) allows the induction of the reporter gene by the various treatments mentioned above; the proximal portion of the promoter between −406 and +24 does not therefore prove to be essential to the activity of the promoter. The fragment −1071 to −406 bp therefore appears to be necessary and sufficient for conferring sensitivity to the inducers studied.

The analysis of the response of the promoter with respect to methyl jasmonate and to UV rays indicates that only the promoter fragment from −956 to +24 bP is functional. Specifically, when the fragment (−1073 to −406) is analyzed outside the context of the total promoter (−1215 to +24 bp), no induction is observed. Several regions therefore exist which are required for the inducibility of the COMT II promoter by methyl jasmonate and UV rays: the region between −956 and −937 bp and also one or more regions located in the more proximal portion of the promoter.

### EXAMPLE 4

### Anti-Infectious Activity of COMT II/Megaspermine in Tobacco

### 4.1. Cloning of a cDNA Encoding β-Megaspermine

The cloning of elicitin genes, such as those of parasiticein and of cryptogein, has shown that these genes encode a pre-protein (Kamoun et al., 1993; Panabières et al., 1995). The coding sequence of these elicitins comprises a 20 amino acid signal peptide which allows them to be secreted into the extra-cellular medium, followed by a 98 amino acid sequence corresponding to the mature protein.

The protein sequence of β-megaspermine, determined beforehand (Kauffmann S., unpublished results), shows strong homology with that of cryptogein (Kamoun et al., 1993). We have put forward the hypothesis that the nucleotide sequences of the corresponding genes could be very close. Primers derived from the nucleotide sequence of the cryptogein gene were synthesized in order to isolate, by PCR, a cDNA encoding β-megaspermine. These primers are located in the sequence encoding the signal peptide and contain the translation initiation codon. Amplification reactions were carried out on the reverse transcripts of Phytophthora megasperma using a sense primer derived from the nucleotide sequence of cryptogein and oligodT as the antisense primer. An amplified fragment of approximately 450 nucleotides was obtained. This fragment was cloned into a bacterial vector in order to be sequenced.

The analysis of the sequences revealed that the clone thus obtained encodes a preprotein comprising a 20 amino acid signal sequence and a 98 amino acid sequence corresponding to β-megaspermine (SEQ ID NO 12). The nucleotide sequence corresponding to the signal peptide exhibits, moreover, 100% identity with that of cryptogein.

The native cDNA of β-megaspermine was fused, firstly, to the COMT II promoter and, secondly, to the 35S promoter. The 1239 bp COMT II promoter was used since it contains all the regulatory elements required for its induction.

### 4.2. Production of Transgenic Tobacco Plants

Nicotiana tabacum Samsun NN tobacco plants were transformed with the two constructs via Agrobacterium tumefaciens. Six primary transformants for each construct were regenerated and self-fertilized. The plants derived from the second generation exhibit a normal phenotype, except for some individuals containing the β-megaspermine gene under the control of the 35S promoter. These plants exhibit a growth delay and also have a relatively undeveloped root system. However, no tissue necrosis recalling that developed by the foliar infiltration and possibly being linked to the expression of the elicitin is observed in the transgenic plants.

### 4.3. Analysis of the β-Megaspermine Expression in the Transgenic Tobacco Plants

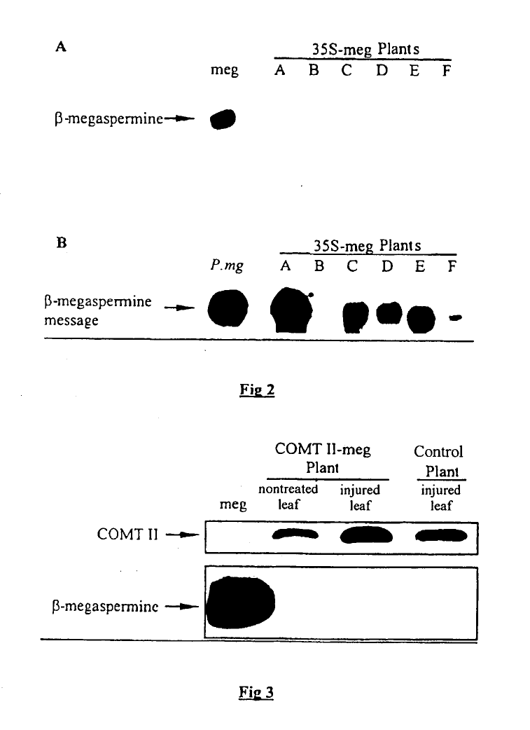

The β-megaspermine expression was analyzed in the leaves of the transgenic plants containing the elicitin cDNA under the control of the 35S promoter, by Western Blot (FIG. 2A) and by Northern Blot (FIG. 2B). Surprisingly, β-megaspermine is undetectable in all the transformants analyzed (FIG. 2A). The level of elicitin transcription was therefore examined in these plants by Northern Blot (FIG. 2B). The transcripts could be detected and the level of expression varies from one transformant to the other. It appears that , in 2 types of plants (E and F), the size of the transcripts is smaller than the size of the complete message. In this case, the absence of protein may be explained by the fact that the cDNA is truncated. For the A, B, C and D plants, the size of the transcripts corresponds to that of the elicitin expressed by the fungus and the transcript content is not insignificant, except for the B plant, which shows a very low proportion of transcripts. The A plants which contain the highest levels of transcripts also show a growth delay. Subsequently, only the transformants having complete β-megaspermine transcripts were tested for their resistance.

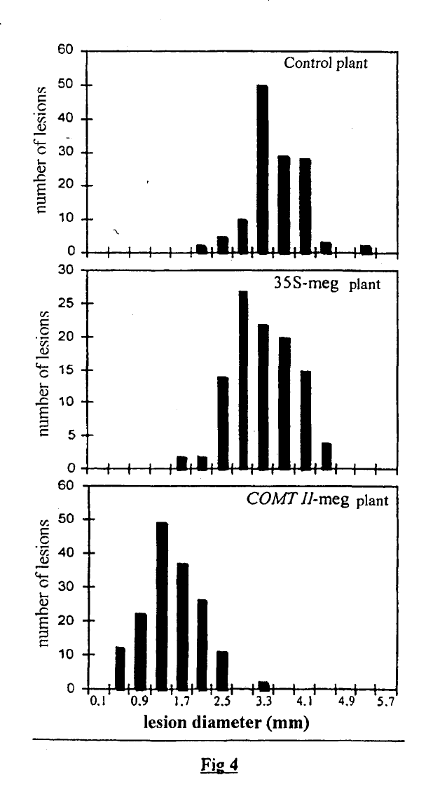

Similarly, the expression of β-megaspermine under the control of the COMT II promoter was analyzed by Western Blot, in the leaves of transgenic tobacco plants (FIG. 3). The expression was studied in nontreated healthy plants in order to determine the basic level of expression of the elicitin and after injury in order to analyze the level of induction obtained. In the nontreated tissues, the level of β-megaspermine is undetectable. On the other hand, the elicitin is detected in very low amounts in the injured tissues. This is due to the activation of the COMT II promoter by the injury. The accumulation of the COMT II was also examined in the same transgenic plants using anti-COMT II antibodies. Surprisingly, the COMT II is detected at a not insignificant level in the nontreated tissues, whereas normally, only very weak COMT II activity is detected in healthy plants which may or may not be transformed with the COMT II::GUS chimeric gene (Collendavelloo et al., 1981; Pellegrini et al., 1993). The COMT II is also produced in greater amounts in the injured tissues compared to the control plants injured in the same way.

The COMT II promoter allows induced expression of the elicitin in the transgenic tobacco plants. This was verified for the 6 transformants. However, the amount of elicitin detected in the plants after induction is very low. The presence of COMT II in the nontreated plants implies that a very low amount of elicitin (undetected by the method used) is produced in the healthy plant and that this synthesis is sufficient to induce the endogenous COMT II gene. In addition, it would appear that induced expression of the elicitin during the injury also allows stronger induction of the endogenous COMT II gene.

Moreover, the analysis of the β-megaspermine on denaturing gel shows that, by electrophoresis, it does not migrate as far as purified mature β-megaspermine (FIG. 3). This difference in migration was evaluated at 3 kD, which corresponds to the size of the signal peptide. This result appears to indicate that the signal peptide was not cleaved. However, it is possible that the mature protein is produced in smaller amounts by the plant cells and that this amount is below the threshold of detection on this Western Blot.

The study of the β-megaspermine expression in transgenic tobacco plants appears to indicate that constitutive expression of elecitin is not tolerated by plants and, consequently, does not allow it to be accumulated in plant cells. Only induced expression allows β-megaspermine synthesis at a detectable level. These results may be linked to the toxic nature of the protein.

### 4.4. Analysis of the Level of Resistance of the Transgenic Plants With Respect to Various Pathogenic Agents

### Antiviral Resistance

a) Resistance with Respect to the Tobacco Mosaic Virus (TMV)

The transformed tobacco plants contain the resistance gene N and therefore react through an HR when TMV is inoculated. In these experiments, resistance to the virus is quantified by measuring the size of the lesions 7 days after infection, when they have reached their virtually definitive size. The greater the resistance developed by the plant, the smaller the size of the lesions, thus illustrating a greater containment of the pathogen at the site. of infection. Since the COMT II gene promoter is strongly induced around the lesions during the HR to TMV, it should allow strong expression of β-megaspermine at the site of infection. In order to verify this, the transgenic plants a, b, c, d, e and f expressing the elicitin under the control of the COMT II promoter and the transformants A, B, C and D containing the β-megaspermine cDNA under the control of the 35S promoter were inoculated with TMV. The control plants are plants which are transgenic for the “COMT II promoter::GUS” chimeric gene.

Seven days after virosis, the lesions developed on the plants expressing 62 -megaspermine under the control of the 35S promoter do not appear to be different to those of the control plants. On the other hand, smaller lesions are observed on the transgenic plants expressing β-megaspermine under the control of the COMT II promoter, compared to those obtained on the control plants.

A statistical analysis of the size of the lesions was performed by measuring the diameter of 100 to 150 lesions in the control plants and in the plants transgenic for β-megaspermine. FIG. 4 represents the size distribution of the lesions measured on the control plants and for a line expressing the elicitin under the control of the 35S promoter (A plants) and a line expressing the elicitin under the control of the COMT II promoter (b plants). This distribution follows a Gaussian curve which permits statistical analysis of the results. The control plants exhibit a lesion size the mean of which is 3.3±0.5 mm. The mean of the lesion size obtained for the A plants is not significantly different from that of the controls (3.1±0.6 mm); on the other hand, that obtained for the b plants is very much smaller than the controls (1.4±0.6 mm). The results obtained for each independent line are given in FIG. 5 and show that all the transformants (A, B, C and D plants) containing the β-megaspermine cDNA under the control of the 35S promoter exhibit a lesion size which does not differ from that of the controls. On the other hand, all the plants expressing the elicitin under the control of the COMT II promoter exhibit a mean lesion size which is significantly smaller than that of the control plants. This decrease is more or less significant depending on the transformants. A large reduction, of approximately 60%, of the lesion size is obtained for the COMT II::meg transformants a, b and f, and the smallest observed is 36% and corresponds to the COMT II::meg transformant e.

The set of results obtained shows that the plants expressing β-megaspermine under the control of the 35S promoter exhibit a level of antiviral resistance which is equivalent to that of the control plants. On the other hand, an increased resistance to TMV is obtained for the transgenic tobacco plants expressing β-megaspermine under the control of the COMT II promoter. However, not all the transgenic lines exhibit the same level of resistance. This may be linked to different levels of β-megaspermine expression in these various transgenic plants.

b) Resistance to the Lucerne Mosaic Virus (LMV).

We tested the resistance of the COMT II::megaspermine transgenic tobacco plants with respect to another virus, LMV, which infects tobacco systemically. About ten days after virosis, the LMV propagated throughout the plant and produced mosaic symptoms in the noninoculated leaves.

The plants expressing β-megaspermine under the control of the COMT II promoter which were chosen for this test correspond to the b line which exhibits a high level of resistance to TMV. The A plants containing the β-megaspermine cDNA under the control of the 35S promoter were also inoculated , as were control COMT II::GUS plants. For each transgenic line, 5 plants were inoculated. We examined the phenotype of the various plants fifteen days after treatment. At this stage, the mosaic symptoms are well developed on the COMT II::GUS plants. The A transgenic plants show a mosaic similar to the control plants. On the other hand, a decrease in these symptoms is observed in the b transgenic plants.

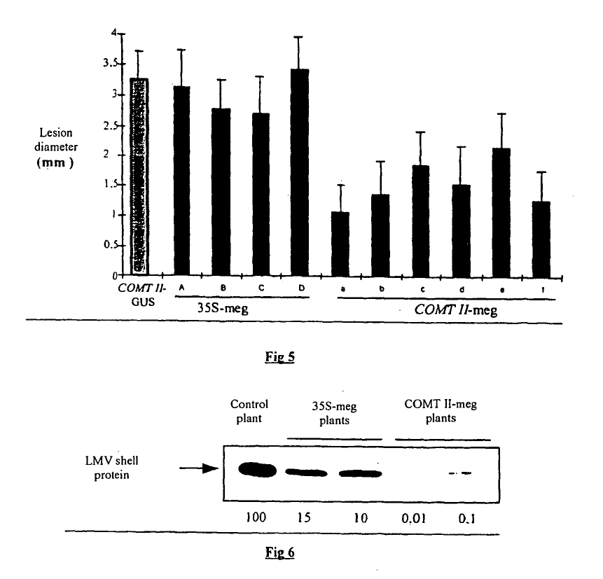

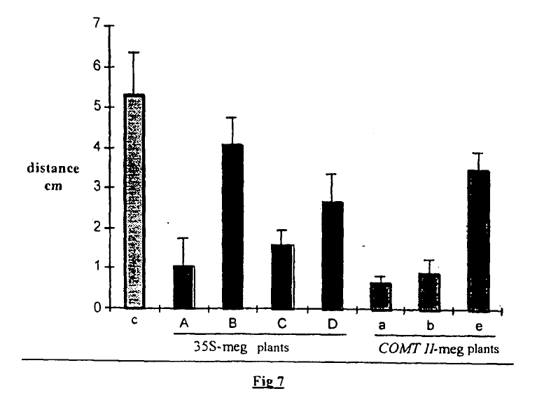

The viral load was examined in the systemic leaves from the same level (3rd leaf above the inoculated leaf). This analysis was carried out by Western Blot using antibodies directed against the protein shell of the virus (FIG. 6). The amount of virus present in the various transgenic plants is evaluated with respect to the amount of virus present in the control plants. The results obtained show that the plants expressing β-megaspermine under the control of the 35S promoter have 10-15 times less virus than the control plants. This decrease in the amount of virus may appear considerable. It has to be remembered, however, that the amounts of virus produced in wild-type plants can vary within the same proportions. On the other hand, the transgenic plants expressing megaspermine under the control of COMT II promoter contain 1000 to 10,000 times less virus that the control plants. This represents a considerable and very significant decrease in the viral load.

These results suggest that only the plants transformed with β-megaspermine under the control of the inducible COMT II promoter are less sensitive to systemic viral infection. This also shows a correlation between the reduction of the infection symptoms and the decrease in the viral load in these leaves.

### Antifungal Resistance

We examined whether the production of elicitins in planta would confer induced resistance with respect to a soil-borne fungus, Phytophthora parasitica var nicotianae. This fungus infects tobacco plants via the roots, invading the root system and then the vascular system, thus causing sclerosis of the conducting vessels. The infection symptoms cause a black rot in the region of the collar. This method of infection is difficult to carry out since it requires a suitable zoospore concentration and corresponds to strict conditions of temperature and humidity. An “artificial” method of inoculation consists in applying, after having decapitated the tobacco plants, the mycelium of the fungus to the severed stem. After 7 days, the stems are removed and the infection symptoms are examined inside the stems.

The inoculation was performed on 7 control COMT II::GUS plants. For each line transgenic for β-megaspermine, five plants were inoculated. The A, B, C and D plants containing the “35S promoter/-megaspermine” construct were tested. For the plants containing the β-megaspermine gene under the control of the COMT II promoter, the a and b lines exhibiting a high increased resistance to TMV were chosen, as well as the e line exhibiting a lower level of resistance.

The symptoms measured in the stems of the control plants progress to reach a mean length of 5.3 cm. Slowing down of the infection symptoms is observed in the stems of the transgenic plants expressing β-megaspermine under the control of the 35S promoter. This decrease is variable from one genotype to the other (FIG. 7). The progression of the fungus is greatly slowed down in the 35S::meg A plants (d=0.8 cm) and less so in the 35S::meg B plants (d=3.8 cm), compared to the control plants (d=5.3 cm). The A plants correspond to the plants which exhibit the highest proportion of β-megaspermine transcripts, while the B plants have a very low content of transcripts encoding the elicitin. This could suggest a correlation between the levels of elicitin transcripts detected (and no doubt the levels of β-megaspermine produced even though the latter remain under the threshold of detection) and the level of antifungal resistance induced.

The progression of the fungus is also greatly slowed down in the stems of the transgenic plants expressing β-megaspermine under the control of the COMT II promoter. We showed, moreover, that the COMT II promoter was induced during infection with P. parasitica. The stems of the COMT II::meg transformants a, b and e are, respectively, infected over a length of 0.7 cm, 1 cm and 3.5 cm. The a and b plants are the most resistant to the infection with P. nicotianae and also correspond to the plants exhibiting the highest resistance to TMV. Thus, it appears that the same relative levels of resistance are detected with the two pathogenic agents tested. It may be assumed that the levels of resistance induced in these plants are linked to the levels of expression of the elicitin. In the knowledge that the protein is difficult to detect by Western blot, analysis of the transcript levels by Northern blot would make it possible to confirm this hypothesis.

These results show that the constitutive or induced expression of β-megaspermine in transgenic tobacco plants makes it possible to confer an increased level of resistance with respect to fungal infection.

### Antibacterial Resistance

We also tested the resistance of the transgenic plants expressing β-megaspermine under the control of the COMT II promoter with respect to a bacterium, Erwinia carotovora. This bacterium is a well known pathogenic agent of the potato and is capable of macerating plant tissues by synthesizing pectinolytic enzymes. However, it can also infect other plants, such as tobacco.

For this test, a suspension of E. carotovora was infiltrated into the leaves of the transgenic COMT II::megaspermine plants and also into the leaves of the control plants (corresponding to the plants transgenic for the chimeric COMT II::GUS gene). A population of 7 plants was inoculated for each of the transformants. Two days after inoculation of the bacteria, 60% of the control plants (4 plants out of 7) exhibit severe infection symptoms, the inoculated leaves are totally macerated and the infection tends to propagate throughout the entire plant. Conversely, 85% of the transgenic plants expressing β-megaspermine under the control of the COMT II promoter (6 plants out of 7) are resistant to the bacterial infection.

These results show that the induced expression of the elicitin greatly increases the resistance with respect to E. carotovora. We have previously shown that the COMT II promoter is inducible by secondary elicitors, among which pectin fragments. Thus, it may be assumed that the pectin fragments produced during the lysis of the plant tissues induce the COMT II promoter and, consequently, the synthesis of β-megaspermine. However, the mechanism which allows the setting up of the antibacterial resistance has yet to be determined.

### Materials and Methods

### Screening of a Genomic Library and Identification of the Clone Corresponding to the Gene Expressed During the Defence Response

A tobacco (Nicotiana tabacum var. Xanthi) genomic DNA library constructed in λ-EMBL3 (Clontech) was screened with a radioactive COMT II cDNA probe (Pellegrini et al., 1993). Six positive genomic clones were isolated after four rounds of purification. These purified clones were tested by PCR in order to identify that which comprises the COMT gene expressed during the hypersensitivity response (HR) of the tobacco leaves to TMV. The 5′ and 3′ primers for the PCR analysis are represented by oligonucleotides 1 and 2 below (SEQ ID NO 4 and SEQ ID NO 5, respectively):

| | |
|---|---|
| Oligo 1: | 5′ CGTTTCGCAA TGTGATTTGA TC 3′ |
| Oligo 2: | 5′ CTCAAAATGA CATCCTTTCA TAC 3; |

They are derived from the untranslated region in 3′ of the COMT II cDNA. The PCR analysis is carried out at 62° C. (theoretical melting temperature) so as to promote specific hybridization. Just one clone amplifies the expected 400 base pair fragment as does the cDNA used as a positive control. The genomic COMT II clone was purified on a Quiagen tip according to the protocol described by the manufacturer and subcloned into the SalI restriction site of the puc 18 plasmid vector.

DNA Sequencing

The DNA sequencing was carried out on denatured double-stranded DNA according to the method of Sanger et al. (1977), using the “rhodamine dye terminator cycle ready” kit with ampliPaq DNA polymerase FS (Perkin-Elmer, P/N402078) and an Applied Biosystems 373 sequencer (Perkin-Elmer). The sequence was determined on both strands with overlaps, using primers synthesized from already determined sequences.

### Analysis of Primer Extension Products

The primer extension reaction was carried out on the total RNA according to the method described in Current Protocols in Molecular Biology (Trienzenberg, 1992). The total RNA isolated from tobacco leaves infected with TMV and from noninfected tobacco leaves (as a negative control) was hybridized to the following oligonucleotide (SEQ ID NO 6), complementary to the COMT II MRNA and labeled at the 5′ end:

Oligo 3: 5′ CTGAAGATGT CAATAGTTGC ATGGC 3′

The extension product was separated on a 6% polyacrylamide gel. The location of the transcription initiation site was determined based on the comigration of that extension products with the sequence ladder of that obtained from the corresponding region of the gene (Sänger et al., 1977).

### Construction of Plasmids

The truncated versions of the COMT II promoter were amplified by PCR using the PSA1 primer in 3′ and PS1, PS2, PS3 and PS4 primers in 5′, represented hereinafter (SEQ ID NO 7 to 11, respectively), producing, respectively, the amplification of the nucleotide fragments with the following lengths: −1215/+24 (novel constructs longer than 600 bp), −420/+24, −313/+24 and −121/+24 (numbering relative to the transcription initiation site).

| | | |
|---|---|---|
| PAS1: | 5′ GGTCTAGAGG GCCTTTTAGA GTGTTTTTGT TAG | 3′ |
| PS1: | 5′ AAAGTCGACC GTCCACCTGT GCCAACAAT | 3′ |
| PS2: | 5′ TGTTTGGTGT TATGCTTCCG TCCT | 3′ |
| PS3: | 5′ AAAAAGCTTT TTTAGGATGG AGTACAGCC | 3′ |
| PS4: | 5′ TTTAAGCTTA AAGAGAACCA GACAATATT | 3′ |

The constructs −1728/+24, −1471/+24, −956/+24, −937/+24, −882/+24, −746/+24, −676/+24, −560/+24 and −435/+24 were obtained with the PAS2 primers in 3′ and, respectively, the PS5, PS6, PS7, PS8, PS9, PS10, PS11, PS12 and PS13 primers in 5′, presented hereinafter (SEQ ID NO 15 to 24, respectively):

| | |
|---|---|
| PAS2: | 5′ CGCGGATCCC CTTTTAGAGT GTTTTTGTTA GGC 3′ |
| PS5: | 5′ ACGCGTCGAC GTTAGGGACA ATCTATAGTG TCAC 3′ |
| PS6: | 5′ ACGCGTCGAC GCTCCGAGGA TTTGGCTGTC GCGG 3′ |
| PS7: | 5′ ACGCGTCGAC GCTGGTTAGG TGAAGTAAAG CATG 3′ |
| PS8: | 5′ ACGCGTCGAC GCATGTTATA TGAGGAAAGT ACG 3′ |
| PS9: | 5′ ACGCGTCGAC GCAGCCAGCA CAAGCAAATT CGC 3′ |
| PS10: | 5′ ACGCGTCGAC GACTTTAACA CACCAACTCC C 3′ |
| PS11: | 5′ ACGCGTCGAC CGGATCTAGA ATTTGGGTTC ATTC 3′ |
| PS12: | 5′ ACGCGTCGAC GTGTATACTC CACGTCTCCG GATAC 3′ |
| PS13: | 5′ ACGCGTCGAC GTTCAATGTT AGGTGTGTTT GG 3′ |

The sequence −1073/−406 which was placed upstream of the minimum 35S promoter was obtained using the PAS3 primer in 3′ and the PS14 primer in 5′ (SEQ ID NO 25 and 26):

PAS3:
5′ CGCGGATCCG CTTAACACCA AACACACCTA ACATTG 3′
PS14:
5′ ACGCGTCGAC CAGTGGTGAG TTTAGCTGTC 3′

The amplification was performed over 30 cycles with an initial step of 4 min at 95° C. and a final step of 5 min at 72° C., using the genomic clone as matrix. Each cycle consists of 1 min at 95° C. followed by 1 min of hybridization and then by 1.5-2 min at 72° C. The hybridization step is carried out at 54° C., 59° C., 55° C. and 50° C. in order to amplify, respectively, the fragments −1215/+24, −420/+24, −313/+24 and −121/+24. For all the other promoter fragments, the hybridization temperature is 60° C. After subcloning in the PGEM-T plasmid (Promega) and amplification in E. coli, the promoter fragment −1215/+24 is digested with SalI and XbaI, the corresponding sites being present in the PS1 and PSA1 primers, respectively. The 420, 313 and 121 base pair promoter fragments are digested with HindiII and XbaI (the site for HindIII is present in the PS2, PS3 and PS4 primers and the XbaI site is present in the PAS1 primer). The fragments −1768/+24, −1471/+24, −956/+24, −937/+24, −882/+24, −746/+24, −676/+24, −560/+24 and −435/+24 are digested with BamH1, present in PAS2 and PAS3, and with Sal1, present in the PS5 to PS14 primers. All the fragments are cloned into the pBI101 plasmid (Qiagen) so as to create a transcriptional fusion with the GUS reporter gene. All the constructs are sequenced in order to confirm their structure and the junction boarders.

The construct consisting of the CaMV 35S promoter upstream of the megaspermine gene was obtained by replacing the GUS gene with the megaspermine gene (SEQ ID NO 12) in the pBi121 plasmid (Clontech).

### Cloning of the Phytophthora megasperma CDNA

10 mg of total RNA from Phytophthora megasperma are used as matrix to synthesize the first strand. The total RNAs are heated for 3 min at 65° C., cooled on ice and incubated for 2 h at 42° C. in 50 ml of reverse transcription buffer (50 mM Tris-HCl, pH 8.3-15 mM MgCl₂-75 mM KCl-1 mM DTT) containing 1 mM of each dNTP, 40 pmol of antisense primer corresponding to oligodT, and 20 U of AMV (avian myeloblastosis virus) reverse transcriptase. The mixture is heated at 94° C. in order to stop the reaction. After precipitation of the reaction mixture with ethanol, the pellet is dissolved in sterile distilled water.

The synthesis of the second strand is initiated by Taq DNA polymerase. 1/20 of the reverse transcription product is used for the amplification by PCR in 50 ml of buffer (10 mM Tris-HCl, pH 8.3-50 mM KC1-1.5 mM MgCl₂-0.01% BSA), 200 mM of dNTP, 0.1 mM of sense and antisense primers and 1 unit of Taq. The sense primer (5′ ATGAACTTCACCGCTCTGCT 3′) derives from the nucleotide sequence of cryptogein, the antisense primer corresponds to oligodT containing the SstI-EcoRI-HindIII restriction sites at its 5′ end. The reaction mixture is heated for 3 min at 94° C. and is then subjected to 30 reaction cycles each comprising 3 steps: 1 min at 94° C., 1 min at 49° C. and 1 min at 72° C. After the final cycle, the elongation is continued for 10 min at 72° C. The amplification product obtained is then cloned into the pGEM vector (Promega) in order to be sequenced.

### Transformation of Plants

The various COMT II promoter/GUS constructs obtained previously in the pBI101 plasmid are introduced into a strain of Agrobacterium tumefaciens GV3101 (pPM6000) (Rossi et al., 1993) by electroporation (Nagel et al., 1990). The tobacco plants (Nicotiana tabacum cv. Samsun NN) are transformed by infiltration of Agrobacterium on 10-day-old plants (Rossi et al., 1993). The plants are regenerated on a Murashige & Skoog (MS) medium (Gibco BRL) supplemented with sucrose (30 g/l for stem formation and 15 g/l for root formation), 6-benzylaminopurine (Serva) (2 mg/ml) and naphthalene acetic acid (Serva) (0.05 mg/ml). Kanamycin (150 mg/ml) is used as a selection agent during the in vitro regeneration and propagation steps. Control plants were prepared by transformation with the empty pBI101 vector or the p min GUS vector (containing the GUS reporter gene under the control of the minimum CaMV 35S RNA promoter). Seven to 20 independent transformants are regenerated for each construct. The primary transformants are self-fertilized and the F1 grains are germinated on an MS medium comprising 300 mg/l of kanamycin.

### Enzymatic Assays

The GUS is histochemically located in the transgenic plants according to the method described by Jefferson et al. (1987). The histochemical reaction is incubated in the dark at 37° C. for 12 hours. The tissues are rinsed, first with a 50 mM phosphate buffer in order to terminate the reaction, and then several times with ethanol from 70% to 90% in order to eliminate the tissue pigmentation. After the histochemical reaction, the tissues are rinsed in 70% ethanol and embedded in a fixing historesin (Jung) for transverse sections of leaves. The historesin blocks are sectioned with a microtome and photographs are taken at a 10 to 40 times magnification using a binocular microscope.

The COMT II and GUS activities were assayed on 100 mg of tissues. The tissue is homogenized in a 100 mM sodium phosphate buffer at pH 7.5 containing 10 mM of DTT, after addition of polyclar AT (Serva) and of quartz. The crude extracts are clarified by centrifugation and by filtration on glass wool. The GUS and COMT II activities are measured on the same crude extracts. For measuring the COMT II activity, an aliquot of the protein extract is added to 1 ml of phosphate buffer comprising 100 μM of catechol and 50 μM of tritiated S-adenosyl-L-methionine (1.5×10⁵ cpm/ml) and incubated for 3 hours at 37° C. The reaction is stopped by adding 100 μl of 9M sulfuric acid. The radioactive reaction product, ferulic acid, is extracted with 5 ml of an NA scintillation solution (Beckman) and the radioactivity is counted on a Beckman LS 9000 machine. The fluorimetric measurement of the GUS activity is performed on the same samples, according to the procedure of Jefferson et al. (1987). The protein content is determined by the Bradford method (1976) using Biorad reagents.

### COMT II-meg and 35S-meg Constructs

Since the PGEM vector (Promega) does not contain cloning sites compatible with those of pBI101, a subcloning step in the Bluescript vector (pSK) was necessary. The BamHI/SstI insert originating from pSK::megaspermine was purified for cloning into the pBI101 binary vector (Clontech).

The pBI101::COMT II-GUS vector previously constructed and corresponding to the promoter −1215/+24 fused to the GUS reporter gene was used to obtain the chimeric COMT II-meg gene. The GUS gene is excised by digesting the binary plasmid with BamHI and SstI. An SnabI site present in the GUS gene makes it possible to cleave the latter and avoid its religation with the binary plasmid since no step for purifying the vector is carried out. The restriction enzymes are heat-inactivated. The digested DNA is extracted with a phenol:chloroform (1:1) mixture and then a chloroform:isoamyl alcohol (24:1) mixture and precipitated with ethanol. The digested DNA is resuspended in sterile water and ligated with the insert.

The pBI121 vector (Clontech) carrying the 35S-GUS gene was used to obtain the chimeric 35S-meg gene. The cloning is carried out according to the method described above.

The constructs were then verified by sequencing.

### Tobacco Plants

The transgenic tobacco plants (Nicotiana tobacum cv. Samsun NN) are cultivated in vitro under a 12h(24° C.)/12h(20° C.) light cycle for 5 weeks after germination. They are propagated on an MS medium with the addition of kanamycin (150 mg/l) as the selection agent. They are then transferred under glass and cultivated in the ground under a 16h/8h light cycle at 22°+2° C.

### Northern Analysis of the Plant RNAs

### Extraction of Total RNAs

The tissues are finely ground in liquid nitrogen and then in 1 to 2 volumes of extraction buffer (0.2 M sodium borate, pH 9-30 mM EGTA-1% SDS-5 mM DTT). The mixture is poured into a tube containing 1 volume of phenol/chloroform (1:1), vortexed and then centrifuged for 10 min at 5 000 g. A second extraction with phenol/chloroform (1:1) is carried out, followed by a third with chloroform/isoamyl alcohol (24:1). The RNAs present in the supernatant are specifically precipitated by adding 1 volume of a solution of LiCl (4 M) and EDTA (10 mM) overnight at 0° C. The entire mixture is centrifuged for 30 min at 10 000 g and the pellet is washed with a solution of LiCl (2 M) and EDTA (5 mM). The total RNAs are taken up in water and again precipitated with 70% ethanol and NaCl (0.2 mM). After centrifugation for 30 min at 10 000 g, the pellet is washed twice with 70% ethanol and then taken up in 50 μl of water. The RNAs are assayed by measuring the absorbence of a diluted solution at 260 (1 OD₂₆₀ unit=40 μg/ml of RNA) and their integrity is verified by migration on a nondenaturing agarose gel stained with ETB.

### Electrophoresis

The RNAs are separated on denaturing agarose gel prepared in 1×MOPS buffer containing 16% of formaldehyde. 1.2% (w/v) agarose gels were used. The RNA samples are denatured for 15 min at 65° C. in the presence of 3 volumes of denaturation solution (10 μl 5×MOPS, 50 μl formamide, 16 μl formaldehyde) per volume of RNA and rapidly cooled in ice. Loading buffer is added (1×MOPS, 50% glycerol, 0.05% bromophenol blue) in a proportion of 1/10 of the volume. After migration in 1×MOPS buffer, the gel can be stained with ETB (0.5 μg/ml) for 1 to 2 min and then thoroughly washed with sterile water. The RNAs are then visualized under UV light.

### “Northern Blot”

The Northern blot technique makes it possible to detect, among the total RNAs, the messages homologous to a radioactive DNA probe. The latter is produced using the Random Priming kit (Amersham), according to the protocol supplied.

10 μg of stem or leaf total RNAs are loaded onto a denaturing 1.2% (w/v) agarose gel and an RNA size marker (Promega) is also loaded at the edge of the gel. After migration, the gel is rinsed with sterile water in order to remove excess formaldehyde. The migration band corresponding to the size marker is cut out, stained with ETB and photographed.

The RNAs are transferred by capillarity for 5 h onto a positively charged nylon membrane (Hybond N⁺, Amersham) with 20×SSC buffer. After transfer, the membrane is rinsed in the 2×SSC buffer and the RNAs are fixed onto the nylon membrane covalently by exposure to ultraviolet light (1 200 J, UV Stratalinker 2400, Stratagene). The membrane is hybridized to a radioactive probe and treated as described in Sambrook et al.

### Western Analysis of Plant Proteins

### Extraction of Foliar Proteins

The extraction can be carried out immediately after harvesting or on frozen samples. 150 g of foliar tissues are ground with a mortar in 0.5 M sodium acetate buffer, pH 5.2, containing 2-β-mercaptoethanol (15 mM) and active charcoal. The buffer volume is adjusted so as to obtain fine and homogeneous ground material. The latter is then filtered over gauze and then centrifuged for 30 min at 15 000 g. The supernatant constituting the crude extract is then analyzed.

### Protein Assay

The protein concentration of the crude extracts is assayed using the Bradford method (1976) in microtitration plates: 200 μl of 1×Bradford reagent (Biorad) are added to 10 μl of an extract to be tested. 2 μl of crude leaf extract are made up to 10 μl with buffer. Each sample is tested three times. After 5 to 10 min, the plate is read on an MR 700 spectro-photometer (Dynatech) with the 5 filter (660 nm). The blank consists of 10 μl of buffer and the values are compared to a standard range prepared with bovine serum albumin (BSA, Sigma).

### Separation of Proteins on Denaturing Polyacrylamide Gel

The analysis on gel makes it possible to determine the molecular mass of the proteins by comparing their relative mobility to those of proteins of known molecular mass. 20% (v/v) of loading buffer (60 mM Tris-HCl, pH 6.8, 5% (v/v) 2-β-mercaptoethanol, 10% (v/v) glycerol, 0.01% (v/v) bromophenol blue and 1% SDS) are added to the protein extract. The samples are heated at 100° C. for 1 min before being loaded. The gels (0.75 mm×7 cm×9 cm) are poured between a glass plate and an alumina plate. The electrophoresis buffer is composed of 192 mM glycine, 25 mM Tris and 0.1% SDS. The vertical migration is carried out in a Hoeffner electrophoresis tank at a strength of 20 mA per gel for a tension of 100 to 160 V.

### Transfer and Immunodetection of the Proteins on Nitrocellulose (Western Blot)

After electrophoretic migration on acrylamide gel, the proteins can be transferred, in liquid medium in the transfer buffer (0.16 M Tris-1.20 M glycine), onto a membrane of the nitrocellulose or nylon PVDF type (Immobilon, Millipore). Before transfer, the gel is equilibrated in the transfer buffer. The PVDF membrane is activated for 1 min in methanol and then also equilibrated in the transfer buffer. The electrotransfer is carried out for 90 min at 150 mA.

The immunodetection was carried out using specific antibodies directed against megaspermine or the LMV shell protein. Revelation is carried out by chemiluminescence with the ECL kit (Amersham).

### Treatment of Plants

Before treatment, the plants are conditioned for a few days in a small air-conditioned cell at 22° C. ±1° C., under a luminosity of 5 000 lux and a D/N photoperiod of 16 h/8 h. These conditions are maintained during infection, with the exception of infection with Erwinia carotovora.

### Inoculation of the Tobacco Mosaic Virus (TMV)

6-week-old tobacco plant leaves cultivated under glass are rubbed using a cotton swab soaked beforehand in a suspension of purified TMV (common strain U1 0.1 to 1 μg/ml) containing an abrasive, celite (10 mg/ml). The leaves of tobacco vitroplants, aged 6 to 7 weeks after planting out, are rubbed using a sintered glass spatula immersed beforehand in a suspension of purified TMV (common strain U1 at 4 μg/ml, filtered over a 0.45 μm membrane) and an abrasive, celite (10 mg/ml). After contact for a few minutes, the leaves are rinsed with water in order to remove excess celite and excess viral particles.

### Inoculation of the Lucerne Mosaic Virus (LMV)

6-week-old tobacco plant leaves are rubbed using a glass spatula soaked in a buffer solution containing the viral RNA (1 to 5 μg/ml) and an abrasive, celite (10 mg/ml). The inoculum per leaf (for one plant) corresponds to 200 ml of buffer solution: 0.04M, Kpi, pH 7.2, 1 ml RNAsin, 1 mM final DTT, Macaloid (0.05%), 0.25 mg/ml total yeast RNA, viral RNA (1 to 5 μg/ml).

### Inoculation of Phytophthora parasitica var. nicotianae

The mycelium of P. p. nicotianae is cultivated on a Petri dish containing a solid medium (oat medium: 100 g of ground oat grains are suspended in 1 l of distilled water. The medium is filtered over gauze. The medium is autoclaved after adding 15 g of agar).

The inoculation was carried out after having decapitated the tobacco plants. The stem of 10-week-old tobacco plants is severed below the apical bud. A disc of P. p. nicotianae mycelium is removed at the periphery of a 7-day culture on oat medium and is placed on the section of the stem. The stem brought into contact with the mycelium disc is encapsulated in a sheet of aluminum in order to prevent the tissues at the site of inoculation drying out too rapidly.

### Inoculation of Erwinia carotovora

The Erwinia carotovora strain is cultured overnight at 28° C. in an LB medium. After centrifugation, the bacterial pellet is taken up in an MgSO₄ solution (10 mM) so as to obtain an approximate bacterial concentration of 1×10⁷ cfu (colony-forming units)/ml. The leaves of 4- to 5-week-old tobacco plants are infiltrated with this bacterial suspension. A single site is inoculated per leaf, by infiltrating 50 ml of the bacterial suspension using a pipetteman. The plants are then placed in small cells under conditions of high humidity and at a temperature of 26° C.±1° C. during the infection.

Treatment with an elicitor: Solutions are infiltrated, using a syringe equipped with a fine needle, in the mesophyl of the leaves. The infiltrated regions are delimited with a felt-tip marker. The infiltrated regions are harvested 16 hours after treatment. The leaves are treated with a solution of β-megaspermine, a protein elicitor purified from a Phytophthora megasperma culture medium (Kauffmann et al., 1993), or of oligosaccharides such as chitin oligomers (100 μg/ml), glucan fragments (200 μg/ml) and pectin fragments (200 μg/ml). The control plants are infiltrated with water.

Injury: Totally developed leaves are injured with a hemostat or pierced with needles. The injured leaves are then harvested 16 or 24 hours after injury for fluorimetric or histochemical analyses.

UV treatment: The upper part of 5-week-old transgenic plants is exposed to UV rays (1=254 nm) for 10 minutes and then the plants are placed in the dark until the tissues are collected 16 hours after treatment.

Chemical treatments: (1) Solutions of SA (1 mM) or of INA (50 mM) are infiltrated into tobacco leaves using.the protocol described for the treatment with elicitors. (2) Under identical culture conditions, solutions of SA (10 mM), INA (1 mM) or BTH (50 mM) are sprayed onto the transgenic plants. The tissues are recovered 16 hours after treatment. Plants treated with water are used as a control. (3) Seven-week-old plants or leaves of vitroplants kept alive on water are transferred into transparent, hermetically sealed dishes and subjected to an atmosphere of 3.5 mM of MeJa (Serva). The tissues are removed at various times after treatment. The control plants are placed in the same dishes without MeJa.

References:

Ausubel, F. M., Brent, R., Kingston, R. E., Moore, D. D., Seidman, J. G., Smith, J. A., Struhl, K. (1998) Current protocols in molecular biology. John Wiley & Sons. Bradford M. M. (1976). A rapid and sensitive method for the quantitation of microgram quantities of protein utilising the principle of protein-dye binding, Anal. Biochem. 72, 248-254.

Collendavelloo J, Legrand M, Geoffroy P, Barthelemy J & Fritig B (1981). Purification and properties of the three o-diphenol O-methyltransferases of tobacco leaves.

Phytochemistry 20, 611-616.

Jefferson R. A., Kavanagh T. A. & Bevan M. W. (1987). GUS fusions: b-glucuronidase as a sensitive and versatile gene fusion marker in higher plants. EMBO J. 6, 3901-3907. Kamoun S, Young M, Glascock CB & Tyler BM (1993). A gene encoding a host-specific elicitor protein of Phytophthora: host specificity and induction of resistance to bacterial and fungal pathogens. Mol. Plant Microbe Interact. 6, 15-25.

Kauffmann S., Baillieul F., Genetet I., Kopp M. & Fritig B. (1993). Two proteins secreted by Phytophthora megasperma elicit and defense-related responses in tobacco. In Mechanisms of plant defense responses, B. Fritig and M. Legrand, Dordrecht: Kluwer Academic Publishers, 140-143.

Nagel R., Eliott A., Masel A., Birch R. G. & Manners J. M. (1990). Electroporation of binary Ti plasmid vector into Agrobacterium tumefaciens and Agrobacterium rhizogenes. FEMS Micro\acute{b}iol. Lett. 67, 325-328. Panabières F, Marais A, Berre JYL, Penot I, Fournier D & Ricci P (1995).

Characterization of a gene cluster of Phytophthora cryptogea which codes for eliciting, proteins inducing a hypersensitive-like response in tobacco. Mol. Plant Microbe Interact. 8, 1995.

Pellegrini L., Geoffroy P., Fritig B. & Legrand M. (1993). Molecular cloning and expression of a new class of ortho-diphenol-O-methyltransferases induced in tobacco (Nicotiana tabacum L.) leaves by infection or elicitor treatment. Plant Physiol. 103, 509-517. Rossi L., Escudero J., Hohn B. & Tinland B. (1993). Efficient and sensitive assay for T-DNA dependant transient gene expression. Plant Mol. Biol. Rep. 12, 220-229.

Sambrook, J., Fritsch, E. F. and Maniatis, T. (1989) Molecular cloning: a laboratory manual. Cold Spring Harbor, New York: Cold Spring Harbor Laboratory Press. Sänger F., Nicklens S. & Coulson A. R. (1977). DNA sequencing with chain-terminating inhibitors. Proc. Natl. Acad. Sci. USA. 74, 5463-5467.

Trienzenberg S. J. (1992). Primer extension. In Current Protocols in Molecular Biology. J. Wiley & Sons eds. 20.4.8.1

1. A nucleic acid fragment comprising an inducible plant promoter, characterized in that said inducible promoter consists of the promoter of a plant class II O-methyltransferase (COMT II) gene.

2. The nucleic acid fragment as claimed in claim 1, characterized in that it consists of a plant COMT II promoter.

3. The nucleic acid fragment as claimed in either of claims 1 and 2, characterized in that the promoter is activated by injuries, viral infections, attacks from UV rays, chemical attacks or attacks by a pathogen, an insect or a nematode.

4. The nucleic acid fragment as claimed in one of claims 1 to 3, characterized in that the plant COMT II is a plant COMT which is not expressed in untreated healthy plants but which is expressed subsequent to a mechanical or chemical attack or an attack by a pathogen, an insect or a nematode.

5. The nucleic acid fragment as claimed in one of claims 1 to 4, characterized in that the plant is a monocotyledon plant or a dicotyledon plant in particular chosen from rice, wheat, barley, sunflower, maize, tobacco, rapeseed, soybean or Arabidopsis thaliana.

6. The nucleic acid fragment as claimed in claim 5, characterized in that the plant is a dicotyledonous plant, preferably tobacco.

7. The nucleic acid fragment as claimed in one of claims 1 to 6, characterized in that the promoter comprises a sequence in 5′ of the translation initiation site, or start codon (ATG), of the coding sequence of the COMT II, which allows the transcription and the expression of said coding sequence.

8. The nucleic acid fragment as claimed in claim 1 to 7, characterized in that the promoter comprises a sequence of more than 600 nucleotides upstream of the COMT II ATG, preferably of more than 1000 nucleotides upstream of the ATG, more preferentially of more than 1200 nucleotides upstream of the ATG.

9. The nucleic acid fragment as claimed in one of claims 1 to 8, characterized in that the promoter comprises a transcription initiation site located less than 100 nucleotides upstream of the ATG, advantageously approximately 90 nucleotides upstream.

10. The nucleic acid fragment as claimed in one of claims 1 to 9, characterized in that the 3′ end of the promoter is located between the transcription initiation site and the ATG.

11. The nucleic acid fragment as claimed in claim 10, characterized in that the 3′ end of the promoter is located between 10 and 50 nucleotides downstream of the transcription initiation site, more preferentially between 20 and 40 nucleotides downstream, even more preferentially between 20 and 30 nucleotides downstream.

12. The nucleic acid fragment as claimed in one of claims 1 to 11, characterized in that it consists of the tobacco COMT II promoter defined by the nucleotide sequence upstream of the ATG represented by sequence identifier 1 (SEQ ID NO 1), the sequences capable of hybridizing selectively to said sequence and the homologous sequences.

13. The nucleic acid fragment as claimed in claim 12, characterized in that it consists of the sequence between nucleotides 557 and 1796 of sequence identifier No. 1.

14. A chimeric gene (or expression cassette) which is functional in plant cells and the plants comprising, in the direction of transcription, a regulatory sequence in 5′, a coding sequence and a regulatory sequence in 3′, characterized in that the regulatory sequence in 5′ comprises the nucleic acid fragment as claimed in one of claims 1 to 13.

15. The chimeric gene as claimed in claim 14, characterized in that the coding sequence comprises a coding sequence for a reporter gene or a coding sequence for a protein of interest.

16. The chimeric gene as claimed in claim 15, characterized in that the protein of interest is a protein which confers on the plants properties of resistance to diseases or to insects.

17. The chimeric gene as claimed in claim 16, characterized in that the protein or peptide of interest is chosen from fungal elicitor peptides, in particular eliciting.

18. The chimeric gene as claimed in claim 17, characterized in that the fungal elicitor peptide is megaspermine.

19. The chimeric gene as claimed in claim 18, characterized in that megaspermine is represented by sequence indentifier No. 13 (SEQ ID 13).

20. The chimeric gene as claimed in claim 19, characterized in that it comprises the DNA sequence represented by sequence identifier No. 14 (SEQ ID 14).

21. A chimeric gene which is functional in plant cells and plants, characterized in that it comprises, in the direction of transcription, a regulatory sequence in 5′ comprising an inducible promoter, a coding sequence for an elicitor and a regulatory sequence in 3′.

22. The chimeric gene as claimed in claim 21, characterized in that the elicitor is defined in claims 17 to 19.

23. The chimeric gene as claimed in either of claims 21 and 22, characterized in that the inducible promoter is chosen from the promoters of phenylalanine ammonia lyase (PAL), of HMG-CoA reductase (HMG), of chitinases, of glucanases, of proteinase inhibitors (PIs), of PR1 family genes, of nopaline synthase (nos) or the vspB gene, the HMG2 promoter, the apple beta-galactosidase (ABG1) promoter or the apple aminocyclopropane carboxylate synthase (ACC synthase) promoter.

24. A cloning and/or expression vector for transforming plant cells or plants, characterized in that it contains at least one chimeric gene as claimed in claims 14 to 23.

25. A method for transforming plant cells, characterized in that it consists in integrating into the genome of said plant cells at least one chimeric gene as claimed in one of claims 14 to 23.

26. A transformed plant cell, characterized in that it comprises a chimeric gene as claimed in one of claims 14 to 23.

27. A transformed plant, characterized in that it comprises a chimeric gene as claimed in claims 14 to 23.

28. A plant, characterized in that it contains transformed cells as claimed in claim 26 or obtained using the method as claimed in claim 25.

29. The plant as claimed in claim 28, characterized in that it is regenerated from transformed cells as claimed in claim 19 or obtained using the method as claimed in claim 18.

30. A plant derived from culturing and/or crossing regenerated plants as claimed in claim 29.

31. The plant as claimed in one of claims 27 to 30, characterized in that it is of the monocotyledon type, in particular a cereal, sugar cane, rice or maize, or of the dicotyledon type, in particular tobacco, soybean, rapeseed, cotton, sunflower, beetroot or clover.

32. A grain of plants as claimed in one of claims 27 to 31.


SEQUENCE LISTING (26 sequences)

SEQ ID NO: 1 - DNA, 1863 bp, Nicotiana tabacum
  misc_signal (667)...(672): Inverted W box
aaagttagggacaatctatagtgtcacaaagttgcttatggcttttggttcagataaagaaaaagaacagcattttaatttgtgaagattagtctgagcagaatttcattgtatctagaaagaaattgaaaaaagaaatattctatttcactattatgttaggtgcaactatatcatcaccatggaaaagccggagtaaaaagagaacgtagaggagatttcatgatttgattgagaatataatatattatttttttgtaattccacacaaagattaagaaaatgatctgatcaatgatggctccgaggatttggctgtcgcgggaactatgacattaatataaatttgtcgctgcctataaagaccctatctatctatctatctatctatatatatatatatatatatatatatatatatatatatatatatatatatatatatatatatatatataagcgctaatatttgattattttttaaaaatatttataagtatatatgaaatttttgacgaaatttttgtgtgaccgtgacccctcaacctatagtgtgcgtccacctgtgccaacaatatagagacaatttgctcgtatagtcagaaagagtgttttactttttagttgctttttagtgaatctactcggtataaagttaaattagtgggtcaataagtcgggtgaatagttaaagaaaacagtggtgagtttagctgtcaaataatttcttctttttcttgttttcacattagaaatcaaaataaaacacaagctttttgtatttattttaacacaagctaattatatgtttatatgctggttaggtgaagtaaagcatgttatatgaggaaagtacgaagaaaatgtgccaattgtcgtgtacagcaaagcagccagcacaagcaaattcgcacttgataagtggctaagtccactttctagtggacctagtggttcactaacttttaccaaaaaggcaataatttgcaattcaaaaagaaaaaaggaaaaaagaaaactagacagactttaacacaccaactcccacaggaagcaacaatgcaactcacaaaaggaaaccgagtttttccgcgacggatctagaatttgggttcattctttacgctttttcgtattaaactcattatatttgtataattatgggtttatattttttatttattgtaatttttgtaaaattttatatataagtgtatactccacgtctccggatactacattagcctctagggttcttaatactcttgttaaattgtccaggctccaaacgcatgttcgtttcaattttaacggatgtttccgaacaactccaaatgttcaatgttaggtgtgtttggtgttaagcttccgtcctaggttaatagaatagataattgttgtttcttatatagttttgaacaatcgtcgccataaactaatttttaggatggaagctaatttttaggatggagtacagcctaaggttaaaatataactataaaaaatatccataaaaggtgaaatttaattagtaacatgaaaagataaaactagtgttatcggtcaaactttcaaaagagaaagaaataactagacaaacttcaacaaccaacctgcccaacatgctactgtgcaattgaaaaataaacaaaagagaaccagacaatatttcaaccaatattccatcaagaaaaccaattatgacaattcttaaccaaagtcacaactaacacttataaaaagcactaactcaactgtacatgattgtgaagcctaacaaaaacactctaaaaggaaaagactacgagaataattacactacaactcttatagctaattcttgtctcaagattttcagctatg

SEQ ID NO: 2 - DNA, 5371 bp, Nicotiana tabacum
  promoter (1)...(1860)
  misc_signal (1772)...(0): Transcription origin
aaagttagggacaatctatagtgtcacaaagttgcttatggcttttggttcagataaagaaaaagaacagcattttaatttgtgaagattagtctgagcagaatttcattgtatctagaaagaaattgaaaaaagaaatattctatttcactattatgttaggtgcaactatatcatcaccatggaaaagccggagtaaaaagagaacgtagaggagatttcatgatttgattgagaatataatatattatttttttgtaattccacacaaagattaagaaaatgatctgatcaatgatggctccgaggatttggctgtcgcgggaactatgacattaatataaatttgtcgctgcctataaagaccctatctatctatctatctatctatatatatatatatatatatatatatatatatatatatatatatatatatatatatatatatatatataagcgctaatatttgattattttttaaaaatatttataagtatatatgaaatttttgacgaaatttttgtgtgaccgtgacccctcaacctatagtgtgcgtccacctgtgccaacaatatagagacaatttgctcgtatagtcagaaagagtgttttactttttagttgctttttagtgaatctactcggtataaagttaaattagtgggtcaataagtcgggtgaatagttaaagaaaacagtggtgagtttagctgtcaaataatttcttctttttcttgttttcacattagaaatcaaaataaaacacaagctttttgtatttattttaacacaagctaattatatgtttatatgctggttaggtgaagtaaagcatgttatatgaggaaagtacgaagaaaatgtgccaattgtcgtgtacagcaaagcagccagcacaagcaaattcgcacttgataagtggctaagtccactttctagtggacctagtggttcactaacttttaccaaaaaggcaataatttgcaattcaaaaagaaaaaaggaaaaaagaaaactagacagactttaacacaccaactcccacaggaagcaacaatgcaactcacaaaaggaaaccgagtttttccgcgacggatctagaatttgggttcattctttacgctttttcgtattaaactcattatatttgtataattatgggtttatattttttatttattgtaatttttgtaaaattttatatataagtgtatactccacgtctccggatactacattagcctctagggttcttaatactcttgttaaattgtccaggctccaaacgcatgttcgtttcaattttaacggatgtttccgaacaactccaaatgttcaatgttaggtgtgtttggtgttaagcttccgtcctaggttaatagaatagataattgttgtttcttatatagttttgaacaatcgtcgccataaactaatttttaggatggaagctaatttttaggatggagtacagcctaaggttaaaatataactataaaaaatatccataaaaggtgaaatttaattagtaacatgaaaagataaaactagtgttatcggtcaaactttcaaaagagaaagaaataactagacaaacttcaacaaccaacctgcccaacatgctactgtgcaattgaaaaataaacaaaagagaaccagacaatatttcaaccaatattccatcaagaaaaccaattatgacaattcttaaccaaagtcacaactaacacttataaaaagcactaactcaactgtacatgattgtgaagcctaacaaaaacactctaaaaggaaaagactacgagaataattacactacaactcttatagctaattcttgtctcaagattttcagctatggaatcctcaaccaaaagccaaataccaacacaatcagaagaagagcgtaactgcacatatgccatgcaactattgtcatcttcagtcctcccctttgtgttgcattcaacaattcaattggaagtttttgagatattagccaaatctaatgacactaaactttctgcttctcaaattgtttctcaaattcctaactgcacaaaacctgaagcacctactatgttaaataggatgctttatgtcttggctagttactccttgtttacttgttccattgttgaagatgaaaaaaataatgggggccaaaaaagagtgtatggtttgtcacaagtgggaaaattctttgttaaaaatgaaaatggtgcatcaatggggccacttttggctttgcttcaaaataaagtattcataaacagctggtaagttttgtcctactgtgtattctttttgcagtggctgtattgattggttgcctttttcacaagacaagattcttaagttttattacttgtcgatttatgttagtcgtatgtgctagtgttattattctccatctgatccttttattggtcactttacctaaaaatattgttacaaaacatttgtccttctagaaaatcaggtattattaatttttcaattccatctttattactccaatagtgaatatggttattaattagtgttttaaggaagatgtaaggataatttaatcaaataggatttattattaatgttgtcaaagattctggtggatggatcggagaaaatttcttcatcttaatcagagtttgatgttcgagccacaggaatgaatttgtttttaatagggagtattttctctttgaatagaccttacacaataaaaggacaacccggtacactaagcttccgttatgcgcggggttcggggaaaggaccgcatcaccaggtctattgtacgcagcgttacccaacgtgaatctaaattaatgagactaaaaaatggaacccaacaccagtgaaaaccaaaaaaagaagcaaactttagtggatggcttggaaagatctttcttcttgaataacttggagcgctatatattaaggcgtcgcagccgttagatactttcaagaagaaagctaaaaaatgttttaaagttacggcgctagaataatgaaatttctctatatatataattcaaaagttaataatttattctcttaacttaaatctatattataaaactatattaagtaacttctgcctaatttataatatacaactaatgttttgagaaaacaaaataacaacaacatcaaacccaatgaaatcccacaagtagagtttggggaggatagtgtgtacggagaccttacccctaccttataaagttaaagaggctgttttcgaaagactctcggctcaagaacattaaaaatttgagaaaacaaaatataaattcaaaacctatattaagtttataatccatggtatattatattggcttagtaatctgaaatgaaagatttatgtttgactcctctaaacttgtttttaatgcaaaagaggcacaacatatatattataagtatctttttttggtttcccactgtggccgctaaattcggattcgctggaagtgtcacattgttggagatgggggcaacgctcacaacaaagacgattctataattagtgttcgaacctgaaattttagttaaagataaagaagtacttaccataatggtagatatgatcatatctgactctctttctaatttcaaattacaggtttgaactaaaagatgcagttcttgaaggaggagttccatttgacagggtacacggtgtgcatgcatttgaatatccaaaatcggacccaaaattcaatgatgttttcaacaaggcaatgatcaatcacacaactgtagtcatgaaaaaaatacttgaaaattacaaaggttttgagaaccttaaaactttggttgatgttggaggtggtcttggagttaacctcaagatgattacatctaaataccccacaattaagggcactaattttgatttgccacatgttgttcaacatgccccttcctatcctggtaccttctctcgttcttattttgttgtttattatatttacttcgatcatcaggtctaggtctgtcaagttaaattcgttctcaaaaaagtttataaaggttttgaactccatcacctattgctttaggattttgagttgtatgctctgagtcttgcgcatggtatcatagtcaatttatttaagctcgttattgcacttgtgaattctattatataaggagtaagcctaccaaaaaggagcgaaaatattttccaaaactctttttaaaccttcctcaccccattcccctctcccctctcccccaacaccacccaccaccccaactcccccgtcttagtttttttatttatcctggactttcttatattttatgctttcctttaattgaactcttgtaactaaaccatttgccccccaccctatagtgtttgcctaaattttatatttttcaaaataatattttctatttactaattaaacattagaaaatatttttcggatttttttccactcaccaaccaagcatgggaaaatagtgataaaactactcatttttcaaaataatattttcaaggaaaacattttcctttataccaaatacccttactcttgtatacaaatcttcatgtcgatgatcttgcaatatatatacatgtatatgtatgatttgataaaccacatgaacaaaatggttgagctctgcgaattgtgatatatgatttgcttatgtgttgtgcactatcaattacttaaattaaacttcatctaataatattgcaggggtggaacatgttgggggagatatgtttgaaagtgttccagaaggagatgctatttttatgaagtggattcttcatgactggagtgatagtcacaacctcaagttgctaaagaactgctacaaggctctaccagacaatggaaaggtgattgttgttgaggccattttaccagtgaaaccagacattgacaccgcagtggttggcgtttcgcaatgtgatttgatcatgatggctcaaaatcctggaggcaaagagcgatcggaagaggagtttcgagccttggctactgaagctggattcaaaggcgttaacttaatatgttgtgtctgtaatttttgggtcatggaattctgcaagtagatttctactgtacattgagtttctactactcttgagtatccatttatggcaatctgggactggaattgcagcttagtccagattgaacattgatattcctaataatatttctattatttcccttgtttatttctcttgtatgaaaggatgtcattttgagtattgataatcatgttctctaggacagaaattgtaactttgtccaactttattgatattcctagtaagatttatatgacatgtgtctctggtttgagaagagtttcaatatctacagacggg

SEQ ID NO: 3 - DNA, 1095 bp, Nicotiana tabacum
  CDS (1)...(1095)
atggaatcctcaaccaaaagccaaataccaacacaatcagaagaagagcgtaactgcacatatgccatgcaactattgtcatcttcagtcctcccctttgtgttgcattcaacaattcaattggaagtttttgagatattagccaaatctaatgacactaaactttctgcttctcaaattgtttctcaaattcctaactgcacaaaacctgaagcacctactatgttaaataggatgctttatgtcttggctagttactccttgtttacttgttccattgttgaagatgaaaaaaataatgggggccaaaaaagagtgtatggtttgtcacaagtgggaaaattctttgttaaaaatgaaaatggtgcatcaatggggccacttttggctttgcttcaaaataaagtattcataaacagctggtttgaactaaaagatgcagttcttgaaggaggagttccatttgacagggtacacggtgtgcatgcatttgaatatccaaaatcggacccaaaattcaatgatgttttcaacaaggcaatgatcaatcacacaactgtagtcatgaaaaaaatacttgaaaattacaaaggttttgagaaccttaaaactttggttgatgttggaggtggtcttggagttaacctcaagatgattacatctaaataccccacaattaagggcactaattttgatttgccacatgttgttcaacatgccccttcctatcctggggtggaacatgttgggggagatatgtttgaaagtgttccagaaggagatgctatttttatgaagtggattcttcatgactggagtgatagtcacaacctcaagttgctaaagaactgctacaaggctctaccagacaatggaaaggtgattgttgttgaggccattttaccagtgaaaccagacattgacaccgcagtggttggcgtttcgcaatgtgatttgatcatgatggctcaaaatcctggaggcaaagagcgatcggaagaggagtttcgagccttggctactgaagctggattcaaaggcgttaacttaatatgttgtgtctgtaatttttgggtcatggaattctgcaagtag

SEQ ID NO: 4 - DNA, 22 bp, Artificial Sequence
  Synthetic Oligonucleotide No. 1
cgtttcgcaatgtgatttgatc

SEQ ID NO: 5 - DNA, 23 bp, Artificial Sequence
  Synthetic Oligonucleotide No. 2
ctcaaaatgacatcctttcatac

SEQ ID NO: 6 - DNA, 25 bp, Artificial Sequence
  Synthetic Oligonucleotide No. 3
ctgaagatgtcaatagttgcatggc

SEQ ID NO: 7 - DNA, 33 bp, Artificial Sequence
  Synthetic Oligonucleotide No. PAS1
ggtctagagggccttttagagtgtttttgttag

SEQ ID NO: 8 - DNA, 29 bp, Artificial Sequence
  Synthetic Oligonucleotide No. PS1
aaagtcgaccgtccacctgtgccaacaat

SEQ ID NO: 9 - DNA, 24 bp, Artificial Sequence
  Synthetic Oligonucleotide No. PS2
tgtttggtgttatgcttccgtcct

SEQ ID NO: 10 - DNA, 29 bp, Artificial Sequence
  Synthetic Oligonucleotide No. PS3
aaaaagcttttttaggatggagtacagcc

SEQ ID NO: 11 - DNA, 29 bp, Artificial Sequence
  Synthetic Oligonucleotide No. PS4
tttaagcttaaagagaaccagacaatatt

SEQ ID NO: 12 - DNA, 354 bp, Phytophthora megasperma
  CDS (1)...(354)
  CDS (1)...(60): Preprotein
atgaacttcaccgctctgctcgctgccgtcgccgccgccttggtcggatctgccaacgccaccgcgtgcaccgccacccagcagaccgctgcgtacaagacactcgtgagcatcctgtcggacgcgtcgttcaacaagtgctctacggattcgggctactccatgctgacggccaaggccctccccaccacggcgcagtacaagctcatgtgcgcgtccacggcatgcaacaccatgatcaagaagatcgtgacgctgaacccgcccaactgcgacctgacggtgcccacgagcggcctggtgctcaacgtgtactcgtacgcgaacggcttctcggacaagtgctcgtcgctg

SEQ ID NO: 13 - DNA, 294 bp, Phytophthora megasperma
  CDS (1)...(294)
accgcgtgcaccgccacccagcagaccgctgcgtacaagacactcgtgagcatcctgtcggacgcgtcgttcaacaagtgctctacggattcgggctactccatgctgacggccaaggccctccccaccacggcgcagtacaagctcatgtgcgcgtccacggcatgcaacaccatgatcaagaagatcgtgacgctgaacccgcccaactgcgacctgacggtgcccacgagcggcctggtgctcaacgtgtactcgtacgcgaacggcttctcggacaagtgctcgtcgctg

SEQ ID NO: 14 - DNA, 1620 bp, Artificial Sequence
  promoter (1)...(1263): COMTII promoter
cgtccacctgtgccaacaatatagagacaatttgctcgtatagtcagaaagagtgttttactttttagttgctttttagtgaatctactcggtataaagttaaattagtgggtcaataagtcgggtgaatagttaaagaaaacagtggtgagtttagctgtcaaataatttcttctttttcttgttttcacattagaaatcaaaataaaacacaagctttttgtatttattttaacacaagctaattatatgtttatatgctggttaggtgaagtaaagcatgttatatgaggaaagtacgaagaaaatgtgccaattgtcgtgtacagcaaagcagccagcacaagcaaattcgcacttgataagtggctaagtccactttctagtggacctagtggttcactaacttttaccaaaaaggcaataatttgcaattcaaaaagaaaaaaggaaaaaagaaaactagacagactttaacacaccaactcccacaggaagcaacaatgcaactcacaaaaggaaaccgagtttttccgcgacggatctagaatttgggttcattctttacgctttttcgtattaaactcattatatttgtataattatgggtttatattttttatttattgtaatttttgtaaaattttatatataagtgtatactccacgtctccggatactacattagcctctagggttcttaatactcttgttaaattgtccaggctccaaacgcatgttcgtttcaattttaacggatgtttccgaacaactccaaatgttcaatgttaggtgtgtttggtgttaagcttccgtcctaggttaatagaatagataattgttgtttcttatatagttttgaacaatcgtcgccataaactaatttttaggatggaagctaatttttaggatggagtacagcctaaggttaaaatataactataaaaaatatccataaaaggtgaaatttaattagtaacatgaaaagataaaactagtgttatcggtcaaactttcaaaagagaaagaaataactagacaaacttcaacaaccaacctgcccaacatgctactgtgcaattgaaaaataaacaaaagagaaccagacaatatttcaaccaatattccatcaagaaaaccaattatgacaattcttaaccaaagtcacaactaacacttataaaaagcactaactcaactgtacatgattgtgaagcctaacaaaaacactctaaaaggcctctagaggatccccggggtaccatgaacttcaccgctctgctcgctgccgtcgccgccgccttggtcggatctgccaacgccaccgcgtgcaccgccacccagcaaaccgctgcgtacaaaacactcgtgagcatcctgtcggacgcgtcgttcaacaagtgctctacggattcgggctactccatgctgacggccaaggccctccccaccacggcgcagtacaagctcatgtgcgcgtccacggcatgcaacaccatgatcaaaaaaatcgtgacgctgaacccgcccaactgcaacctgacggtgcccacgagcggcctggtgctcaacgtgtactcgtacccaaacggcttctcggacaagtgctcgtcgctgtaa

SEQ ID NO: 15 - DNA, 33 bp, Artificial Sequence
  Synthetic Oligonucleotide PAS2
cgcggatccccttttagagtgtttttgttaggc

SEQ ID NO: 16 - DNA, 34 bp, Artificial Sequence
  Synthetic Oligonucleotide PS5
acgcgtcgacgttagggacaatctatagtgtcac

SEQ ID NO: 17 - DNA, 34 bp, Artificial Sequence
  Synthetic Oligonucleotide PS6
acgcgtcgacgctccgaggatttggctgtcgcgg

SEQ ID NO: 18 - DNA, 34 bp, Artificial Sequence
  Synthetic Oligonucleotide PS7
acgcgtcgacgctggttaggtgaagtaaagcatg

SEQ ID NO: 19 - DNA, 33 bp, Artificial Sequence
  Synthetic Oligonucleotide PS8
acgcgtcgacgcatgttatatgaggaaagtacg

SEQ ID NO: 20 - DNA, 33 bp, Artificial Sequence
  Synthetic Oligonucleotide PS9
acgcgtcgacgcagccagcacaagcaaattcgc

SEQ ID NO: 21 - DNA, 31 bp, Artificial Sequence
  Synthetic Oligonucleotide PS10
acgcgtcgacgactttaacacaccaactccc

SEQ ID NO: 22 - DNA, 34 bp, Artificial Sequence
  Synthetic Oligonucleotide PS11
acgcgtcgaccggatctagaatttgggttcattc

SEQ ID NO: 23 - DNA, 35 bp, Artificial Sequence
  Synthetic Oligonucleotide PS12
acgcgtcgacgtgtatactccacgtctccggatac

SEQ ID NO: 24 - DNA, 32 bp, Artificial Sequence
  Synthetic Oligonucleotide PS13
acgcgtcgacgttcaatgttaggtgtgtttgg

SEQ ID NO: 25 - DNA, 36 bp, Artificial Sequence
  Synthetic Oligonucleotide PAS3
cgcggatccgcttaacaccaaacacacctaacattg

SEQ ID NO: 26 - DNA, 30 bp, Artificial Sequence
  Synthetic Oligonucleotide PS14
acgcgtcgaccagtggtgagtttagctgtc